(19) United States
(12) Patent Application Publication
(10) Pub. No.: US 2024/0067736 A1
(43) Pub. Date: Feb. 29, 2024

(54) Use of Anti-EGFR/Anti-Met Antibody to Treat Liver Cancer

(71) Applicant: Janssen Biotech, Inc., Horsham, PA (US)

(72) Inventor: Sheri Moores, Wayne, PA (US)

(21) Appl. No.: 18/143,749

(22) Filed: May 5, 2023

Related U.S. Application Data
(60) Provisional application No. 63/338,807, filed on May 5, 2022.

(51) Int. Cl. C07K 16/28; A61K 45/06; A61P 35/00; C07K 16/30

(52) U.S. Cl. CPC C07K 16/2863; A61K 45/06; A61P 35/00; C07K 16/303; A61K 2039/545

(57) ABSTRACT

The present disclosure provides methods of treating liver cancer in a subject in need thereof by administering a therapeutically effective amount of a bispecific anti-epidermal growth factor receptor (EGFR)/hepatocyte growth factor receptor (c-Met) antibody.

### CROSS-REFERENCE TO RELATED APPLICATIONS

This application claims the benefit of U.S. Provisional Patent Application No. 63/338,807, filed May 5, 2022, the disclosure of which is herein incorporated by reference in its entirety.

### REFERENCE TO SEQUENCE LISTING SUBMITTED ELECTRONICALLY

The sequence listing of the present application is submitted electronically via The United States Patent and Trademark Center Patent Center as an XML formatted sequence listing with a file name “JBI6720USNP1SEQLIST.xml”, creation date of May 4, 2023, and a size of 20 kilobytes (KB). This sequence listing submitted is part of the specification and is herein incorporated by reference in its entirety.

### FIELD

The present disclosure relates to methods of treating liver cancer with a bispecific anti-epidermal growth factor receptor (EGFR)/hepatocyte growth factor receptor (c-Met) antibody.

### BACKGROUND

Liver cancer is the 7th most common malignancy and the 3rd leading cause of cancer-related death worldwide, with over 906,000 new primary liver cancer cases resulting in 830,000 deaths globally in 2020 (Sung 2021; Globocan 2020). The major etiologies of liver cancer include chronic hepatitis B virus (HBV) and hepatitis C virus (HCV) infection, alcohol consumption, steatosis, aflatoxin exposure, and diabetes (Sherman 2005; Thomas 2005). Primary liver cancer includes hepatocellular carcinoma (HCC) (comprising 75%-85% of cases) and intrahepatic cholangiocarcinoma (10%-15%), as well as other rare types (Sung 2021).

Patients with HCC are often diagnosed at an advanced stage with distance metastases or with disease that is not amenable to surgery or local treatment. Patients have a poor prognosis, with a 5-year overall survival (OS) rate of 10-18% (Zeng 2018; Siegel 2018). In China, liver cancer is the 4th most common cancer and 2nd leading cause of cancer death with over 410,000 new primary liver cancer cases resulting in 391,000 deaths in 2020 (Zheng 2019; Globocan 2020). China accounts for 45.3% and 47.1% of global new primary liver cancer cases, and liver cancer-related deaths, respectively (Globocan 2020). Chronic HBV infection is the most frequent cause (84%) of HCC in China (Chinese chronic hepatitis B guideline 2019), while in the Western countries chronic HCV, alcoholic cirrhosis and non-alcoholic steatohepatitis (NASH) are the main causes (Medavaram 2018).

Systemic therapies are recommended for HCC patients who have advanced disease (BCLC [barcelona clinic liver cancer] stage C) or who have intermediate-stage disease (BCLC stage B) and progression with transarterial therapies (Villanueva 2019). Sorafenib, a multiple or multi-kinase inhibitor (MKI) was approved in 2007 as first-line targeted therapy for advanced HCC based on the success of SHARP and Asia-Pacific trial, ushering in the era of systemic treatment (Huang 2020; Llovet 2008; Cheng 2009). With low rates of objective tumor responses (2%), however, the clinical benefit of sorafenib remains modest and the investigation of additional novel therapeutics which can target the complex molecular pathogenesis of HCC continued. As first-line treatment, another MKI, was non-inferior to sorafenib in overall survival (OS) (13.6 months vs 12.3 months; hazard ratio [HR] was 0.92) and objective response rate (ORR) (18.8% vs. 6.5%) (Kudo 2018), and has surpassed the use of sorafenib as a front-line therapy in the current treatment of HCC in China. Newly approved atezolizumab (anti-PDL1 antibody) plus bevacizumab (anti-VEGF antibody) in first line treatment shows better OS (19.2 months), progression-free survival (6.8 months) and ORR (30%) than sorafenib (Finn 2020), and has become another frequently utilized front-line option.

The treatment landscape of HCC in second-line setting has been fast evolving since 2017, with the MKI, immunotherapy checkpoint inhibitors and VEGF antibodies regorafenib, nivolumab, pembrolizumab, cabozantinib, and ramucirumab being approved for patients progressed on or after sorafenib (Bruix 2017; Zhu 2018; Abou 2018; Zhu 2019; Meyer 2018). In China, regorafenib, apatinib, and local anti-PD-1 antibodies (camrelizumab and tislelizumab) monotherapy are approved for advanced HCC in the second-line setting. Collectively, the approved second-line monotherapies show modest objective response rates (4-17%) and OS benefits (8.5-15.1 months) (Bruix 2017; Zhu 2018; Abou 2018; Zhu 2019; Meyer 2018). Recently United States (US) Food and Drug administration (FDA) conditionally approved nivolumab plus ipilimumab (anti-CTLA-4 antibody) with its encouraging 32% ORR data in advanced HCC patients previously treated with sorafenib. (Yau 2020; Saung 2021). However, it is only approved in US and most patients experienced adverse events, with any-grade treatment-related adverse events and grade 3-4 treatment-related adverse events (nivolumab 1 mg/kg plus ipilimumab 3 mg/kg every 3 weeks) of 94% and 76%, respectively (Yau 2020; Saung 2021).

Despite the encouraging recent progress with multi-targeted TKIs and immune-based therapies for advanced HCC, several challenges remain. The most important one is the limited efficacy of current treatment options as described above (Bruix 2017; Zhu 2018; Abou 2018; Zhu 2019; Meyer 2018). Another is drug-related adverse events, which often lead to dose reduction, interruption and/or discontinuation (Niu 2021). The third challenge is drug resistance, which remains a major cause of the failure of targeted therapy (Niu 2021). The underlying mechanisms include the intrinsic HCC heterogeneity and clonal evolution, and the lack of reliable biomarkers to identify HCC patients most likely to benefit from targeted and immune therapies (Niu 2021; Rizzo 2021). Considering the high malignancy and heterogeneity of HCC and scarce effective treatment options, there is a great unmet need to develop novel drugs for treatment of advanced HCC.

### SUMMARY

In one aspect, provided herein is a method of treating liver cancer in a subject in need thereof, comprising administering to the subject a therapeutically effective amount of a bispecific anti-epidermal growth factor receptor (EGFR)/hepatocyte growth factor receptor (c-Met) antibody.

In some embodiments, the liver cancer is hepatic cellular carcinoma (HCC).

In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In some embodiments, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

In some embodiments, the bispecific anti-EGFR/c-Met antibody is an IgG1 isotype.

In some embodiments, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments, the bispecific anti-EGFR/c-Met antibody comprises a biantennary glycan structure with a fucose content of about between 1% to about 15%.

In some embodiments, the bispecific anti-EGFR/c-Met antibody is administered intravenously to the subject.

In some embodiments, the bispecific anti-EGFR/c-Met antibody is administered at a dose of between about 350 mg to about 1400 mg. In some embodiments, the bispecific anti-EGFR/c-Met antibody is administered at a dose of about 350 mg, 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg or 1400 mg.

In one embodiment, the bispecific anti-EGFR/c-Met antibody is administered at a dose of 1050 mg.

In one embodiment, the bispecific anti-EGFR/c-Met antibody is administered at a dose of 1400 mg.

In some embodiments, the bispecific anti-EGFR/c-Met antibody is administered subcutaneously or intradermally to the subject. In some embodiments, the bispecific anti-EGFR/c-Met antibody is administered subcutaneously or intradermally at a dose sufficient to achieve a therapeutic effect in the subject.

In various embodiments, the bispecific anti-EGFR/c-Met antibody is administered twice a week, once a week, once in two weeks, once in three weeks or once in four weeks. In some embodiments, the bispecific anti-EGFR/c-Met antibody is administered twice a week, once a week, once in two weeks, once in three weeks or once in four weeks. In some embodiments, the bispecific anti-EGFR/c-Met antibody is administered once a week for four weeks and once in two weeks thereafter.

In various embodiments, the subject has received a prior treatment. In some embodiments, the prior treatment comprises a multi-targeted kinase inhibitor (MKI), an immunotherapy, anti-VEGF/VEGFR therapy, or a combination thereof. In some embodiments, the prior treatment comprises a multi-targeted kinase inhibitor (MKI).

In some embodiments, the multi-targeted kinase inhibitor (MKI) is sorafenib regorafenib, lenvatinib, cabozantinib, apatinib, or a combination thereof.

In some embodiments, the immunotherapy comprises a PD-(L)1 axis inhibitor, or a CTLA-4 inhibitor. In some embodiments, the PD-(L)1 axis inhibitor comprises atezolizumab, nivolumab, pembrolizumab, camrelizumab, tislelizumab, or a combination thereof. In some embodiments, the CTLA-4 inhibitor comprises ipilimumab.

In some embodiments, the anti-VEGF/VEGFR therapy comprises bevacizumab or ramucirumab.

In various embodiments, the subject is treatment naive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts one aspect of the multicenter Phase 2 interventional study described in the Examples.

FIGS. 2A-2B show expression levels pMet and Met in 24 HCC cell lines.

FIG. 3 shows an expression profile of EGFR in 30 HCC cell lines.

FIGS. 4A-4C show in vitro HCC cell growth inhibition by amivantamab. FIG. 4A shows the anti-proliferative activity of amivantamab on NCI-H292 cells. FIG. 4B shows the anti-proliferative activity of amivantamab on MKN45 cells. FIG. 4C shows the anti-proliferative activity of amivantamab on IM95 cells.

FIGS. 5A-5C show in vitro HCC cell growth inhibition by amivantamab. FIG. 5A shows the anti-proliferative activity of amivantamab on SNU398 cells. FIG. 5B shows the anti-proliferative activity of amivantamab on JHH-5 cells. FIG. 5C shows the anti-proliferative activity of amivantamab on MHCC97H cells.

FIGS. 6A-6B show that amivantamab significantly inhibited colony formation in IM95 (FIG. 6A) and JHH-5 (FIG. 6B) cells.

FIGS. 7A-7B shows that amivantamab showed limited effect on colony formation in MKN45 (FIG. 7A) and MHCC97H (FIG. 7B) cells.

FIG. 8 shows the antitumor activity of JNJ-38877618 (CAS Registry No. 943540-74-7) and amivantamab as single agents in subcutaneous MHCC97H xenograft model.

FIG. 9 shows pMet modulation in xenograft tissues after dosing with cMet inhibitors.

FIG. 10 shows the in vivo efficacy of amivantamab in HCC PDX model LI0801.

FIG. 11 shows the in vivo efficacy of amivantamab in HCC PDX model LI1646.

FIG. 12 shows the in vivo efficacy of amivantamab in HCC PDX model LI1098.

FIGS. 13A-13B show the anti-tumor activity of amivantamab in HCC PDX model LI1037. FIG. 13A shows the in vivo efficacy of amivantamab by tumor volume change in HCC PDX model LI1037. FIG. 13B shows the change in body weight in HCC PDX model LI1037.

FIGS. 14A-14B show the anti-tumor activity of amivantamab in HCC PDX model LI0801. FIG. 14A shows the in vivo efficacy of amivantamab by tumor volume change in HCC PDX model LI0801. FIG. 14B shows the change in body weight in HCC PDX model LI0801.

### DETAILED DESCRIPTION

The mesenchymal-epithelial transition factor (cMet or MET) receptor and its ligand hepatocyte growth factor (HGF) plays a critical role in hepatogenesis, hepatohomeostasis and regeneration following injury (Schmidt 1995; Huh 2004). cMet/HGF binding activates intracellular signaling transduction pathways such as PI3K/AKT/mTOR and the MAPK/ERK pathways, thus promoting cell proliferation, survival, morphogenesis and scattering. Dysregulation of cMet/HGF signaling has been implicated in the development and progression of multiple human malignancies including HCC (Birchmeier 2003; Horiguchi 2002; Zhang 2005; Ang 2013). Because of the signaling cross-talk between EGFR and cMet, inhibition of both pathways in combination may limit compensatory pathway activation and improve overall efficacy.

Overexpression and genetic alterations of the epidermal growth factor receptor (EGFR) and MET have been closely linked to tumorigenesis, aggressive progression, and poor prognosis in several cancer types, supporting their importance as therapeutic targets for cancer therapy (Birchmeier 2003; Hynes 2005; Yano 2003). Indeed, agents targeting EGFR (including anti-EGFR antibodies and EGFR tyrosine kinase inhibitors [TKIs]) have been used for treatments in patients with colorectal cancer, non-small cell lung cancer (NSCLC), and head and neck cancer (Hsu 2018; Li 2020; Taberna 2019). Similarly, the recent approval of MET inhibitors in NSCLC with MET Exon14 skipping alteration further expanded the treatment options for biomarker selected patients (Mathieu 2021). However, these two pathways are not currently targeted in the context of HCC. It is reported that approximately 63.2-66.0% and 25.4- 38.8% of patients with HCC have tumors characterized by overexpression of EGFR and MET, respectively (Huang 2019; Buckley 2008). The overexpression of MET often associates with poor prognosis in HCC (Lee 2013; Kondo 2013). Unlike in NSCLC, where the genetic alterations and gene amplification of EGFR and MET drives pathway activation, the activation of EGFR and MET pathways are frequently driven by the canonical ligand-receptor binding in HCC. Indeed, aberrantly high levels of MET ligand, HGF, have been implicated in promoting HCC progression and metastasis (Junbo 1999, Yang 2022). Given the importance of EGFR and MET pathways in HCC, many clinical trials have examined the activities of EGFR or MET targeting agents. However, results have been disappointing so far. For example, erlotinib, an EGFR TKI, combined with bevacizumab did not achieve encouraging outcome in sorafenib-refractory HCC population (irrespective of EGFR status) (Niu 2017; Philip 2012). Similarly, studies evaluating the efficacy of tivantinib (a selective oral MET inhibitor, Daiichi Sankyo) failed to show clinical benefit in MET-high advanced HCC in a 2nd line phase 3 study. (Rimassa 2018).

One possible reason for these outcomes is the redundant signaling pathways that may confer primary or required resistance to the therapeutic intervention against a single receptor target (Zhang Y 2018). EGFR and MET pathways have many overlapping downstream signaling modules, which collectively are pro-tumorigenic and pro-progression (Hung 2014, Zhang YZ 2018). Co-overexpression of EGFR and MET has been reported in 24.5% of HCC patients (Dong 2019). Thus, the suppression of EGFR or MET pathway alone may not be sufficient to block the oncogenic signaling and elicit clinical benefit in HCC. In NSCLC, it is well known that MET pathway activation mediates treatment resistance with EGFR inhibitors. Similarly, activation of MET pathway is also found to mediate resistance to cetuximab (anti-EGFR antibody) in CRC and head and neck squamous cell carcinoma (HNSCC) (Novoplansky 2019). Conversely, EGFR amplification is recently found as a resistance mechanism in NSCLC treated by MET TKI (Recondo 2020). Therefore, targeting both EGFR and MET may provide therapeutic benefits in treating liver cancer such as HCC.

### Definitions

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“Co-administration,” “administration with,” “administration in combination with,” “in combination with” or the like, encompass administration of the selected therapeutics or drugs to a single patient, and are intended to include treatment regimens in which the therapeutics or drugs are administered by the same or different route of administration or at the same or different time.

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent”, “preventing”, “prevention”, or “prophylaxis” of a disease or disorder means preventing that a disorder occurs in subject.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient, decrease or shrinkage of the size of a tumor, arrested or slowed growth of a tumor, and/or absence of metastasis of cancer cells to other locations in the body.

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Cancer” refers to an abnormal growth of cells which tend to proliferate in an uncontrolled way and, in some cases, to metastasize (spread) to other areas of a patient's body.

“EGFR or c-Met or MET expressing cancer” refers to cancer that has detectable expression of EGFR or c-Met or has EGFR or c-Met mutation or amplification. EGFR or c-Met expression, amplification and mutation status can be detected using know methods, such as sequencing, fluorescent in situ hybridization, immunohistochemistry, flow cytometry or western blotting.

“Epidermal growth factor receptor” or “EGFR” refers to the human EGFR (also known as HER1 or ErbB1 (Ullrich et al., Nature 309:418-425, 1984)) having the amino acid sequence shown in GenBank accession number NP_005219, as well as naturally-occurring variants thereof.

“Hepatocyte growth factor receptor” or “c-Met” or “MET” as used herein refers to the human c-Met having the amino acid sequence shown in GenBank Accession No: NP_001120972 and natural variants thereof.

“Bispecific anti-EGFR/c-Met antibody” or “bispecific EGFR/c-Met antibody” refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

“Specific binding” or “specifically binds” or “specifically binding” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 5×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The dissociation constant may be measured using known protocols. Antibodies that bind to the antigen or the epitope within the antigen may, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody binds one antigen or one epitope, a bispecific antibody binds two distinct antigens or two distinct epitopes.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Complementarity determining regions” (CDR) are antibody regions that bind an antigen. CDRs may be defined using various delineations such as Kabat (Wu et al. (1970) J Exp Med 132: 211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton (1996) J Bmol Biol 263: 800-15). The correspondence between the various delineations and variable region numbering are described (see e.g., Lefranc et al. (2003) Dev Comp Immunol 27: 55-77; Honegger and Pluckthun, (2001) J Mol Biol 309:657-70; International ImMunoGeneTics (IMGT) database; Web resources, http://imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Antigen binding fragment” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Recombinant” refers to DNA, antibodies and other proteins that are prepared, expressed, created or isolated by recombinant means when segments from different sources are joined to produce recombinant DNA, antibodies or proteins.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Antagonist” or “inhibitor” refers to a molecule that, when bound to a cellular protein, suppresses at least one reaction or activity that is induced by a natural ligand of the protein. A molecule is an antagonist when the at least one reaction or activity is suppressed by at least about 20%, 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist.

“PD-(L)1 axis inhibitor” refers to a molecule that inhibits PD-1 downstream signaling. PD-(L)1 axis inhibitor may be a molecule that binds PD-1, PD-Ll or PD-L2.

“Low fucose” or “low fucose content” as used in the application refers to antibodies with fucose content of about between 1%-15%.

“Normal fucose” or “normal fucose content” as used herein refers to antibodies with fucose content of about over 50%, typically about over 80% or over 85%.

### Methods of the Disclosure

One aspect of the disclosure provides a method of treating liver cancer in a subject in need thereof, comprising administering to the subject a therapeutically effective amount of a bispecific anti-EGFR/c-Met antibody.

In some embodiments, the liver cancer is hepatocellular carcinoma (HCC), cholangiocarcinoma or hepatoblastoma. In some embodiments, the liver cancer is HCC. HCC often arises in individuals suffering from other underlying liver pathologies, including hepatitis, cirrhosis, and/or aflatoxin B1 poisoning, which cause liver inflammation and chronic injury and regeneration of liver cells. Diagnosis of liver cancer can be achieved by e.g., ultrasonography, fine-needle biopsy, and detection of circulating levels of certain marker proteins, including, for example, alpha-fetoprotein. HCC can be classified into early, intermediate, advanced, and end-stage cancer based on tumor size, number and morphology (e.g., encapsulated or invasive), and liver function.

In some embodiments, the subject has received prior treatment. The prior treatment may include a multi-targeted kinase inhibitor (MKI), an immunotherapy, anti-VEGF/VEGFR therapy, or a combination thereof.

In some embodiments, the prior treatment comprises a multi-targeted kinase inhibitor (MKI). Non-limiting examples of multi-targeted kinase inhibitors (MKIs) include sorafenib regorafenib, lenvatinib, cabozantinib, and apatinib.

In some embodiments, the prior treatment comprises an immunotherapy such as checkpoint inhibitors. In some embodiments, the immunotherapy comprises a PD-(L)1 axis inhibitor, or a CTLA-4 inhibitor. Non-limiting examples of PD-(L)1 axis inhibitors include atezolizumab, nivolumab, pembrolizumab, camrelizumab, and tislelizumab. Non-limiting examples of CTLA-4 inhibitors include ipilimumab.

In some embodiments, the prior treatment comprises an anti-VEGF/VEGFR therapy. Non-limiting examples of anti-VEGF/VEGFR therapy include bevacizumab and ramucirumab. In some embodiments, the subject is treatment naïve.

### Administration

The bispecific anti-EGFR/c-Met antibody may be administered in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used to formulate the bispecific anti-EGFR/c-Met antibody. These solutions are sterile and generally free of particulate matter. They may be sterilized by conventional, well-known sterilization techniques (e.g., filtration). For parenteral administration, the carrier may comprise sterile water and other excipients may be added to increase solubility or preservation. Injectable suspensions or solutions may also be prepared utilizing aqueous carriers along with appropriate additives. For subcutaneous administration, a recombinant human hyaluronidase, such as rHuPH20 (CAS Registry No. 757971-58-7)) may be used. Suitable vehicles and formulations, inclusive of other human proteins, e.g., human serum albumin, are described, for example, in e.g. Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

The mode of administration may be any suitable route that delivers the bispecific anti-EGFR-c-Met antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intratumoral, intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

In some embodiments, the bispecific anti-EGFR/c-Met antibody is administered intravenously.

In some embodiments, the bispecific anti-EGFR/c-Met antibody is administered subcutaneously or intradermally to the subject. The bispecific anti-EGFR/c-Met antibody may be administered subcutaneously or intradermally at a dose sufficient to achieve a therapeutic effect in the subject.

In some embodiments, the bispecific anti-EGFR/c-Met antibody is administered at a dose of between about 140 mg to about 1750 mg. In some embodiments, the bispecific anti-EGFR/c-Met antibody is administered at a dose of between about 350 mg to about 2240 mg. In some embodiments, the bispecific anti-EGFR/c-Met antibody is administered at a dose of between about 350 mg to about 1400 mg.

In some embodiments, the bispecific anti-EGFR/c-Met antibody is administered at a dose of about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1580 mg, about 1590 mg, about 1600 mg, about 1610 mg, 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, 1890 mg, about 1900 mg, about 1910 mg, about 1920 mg, about 1930 mg, about 1940 mg, about 1950 mg, about 1960 mg, about 1970 mg, about 1980 mg, about 1990 mg, about 2000 mg, about 2010 mg, about 2020 mg, about 2030 mg, about 2040 mg, about 2050 mg, about 2060 mg, about 2070 mg, about 2080 mg, about 2090 mg, about 2100 mg, about 2110 mg, about 2120 mg, about 2130 mg, about 2140 mg, about 2150 mg, about 2160 mg, about 2170 mg about 2180 mg, about 2190 mg, about 2200 mg, about 2210 mg, about 2220 mg, about 2230 mg, about 2240 mg, or about 2250 mg.

In some embodiments, the bispecific anti-EGFR/c-Met antibody is administered at a dose of about 350 mg, about 700 mg, about 1050 mg, about 1400 mg, about 1575 mg, about 1600 mg, about 2100 mg, or about 2240 mg. In some embodiments, the bispecific anti-EGFR/c-Met antibody is administered at a dose of about 350 mg. In some embodiments, the bispecific anti-EGFR/c-Met antibody is administered at a dose of about 700 mg. In some embodiments, the bispecific anti-EGFR/c-Met antibody is administered at a dose of about 750 mg. In some embodiments, the bispecific anti-EGFR/c-Met antibody is administered at a dose of about 800 mg. In some embodiments, the bispecific anti-EGFR/c-Met antibody is administered at a dose of about 850 mg. In some embodiments, the bispecific anti-EGFR/c-Met antibody is administered at a dose of about 900 mg. In some embodiments, the bispecific anti-EGFR/c-Met antibody is administered at a dose of about 950 mg. In some embodiments, the bispecific anti-EGFR/c-Met antibody is administered at a dose of about 1000 mg. In some embodiments, the bispecific anti-EGFR/c-Met antibody is administered at a dose of about 1050 mg. In some embodiments, the bispecific anti-EGFR/c-Met antibody is administered at a dose of about 1100 mg. In some embodiments, the bispecific anti-EGFR/c-Met antibody is administered at a dose of about 1150 mg. In some embodiments, the bispecific anti-EGFR/c-Met antibody is administered at a dose of about 1200 mg. In some embodiments, the bispecific anti-EGFR/c-Met antibody is administered at a dose of about 1250 mg. In some embodiments, the bispecific anti-EGFR/c-Met antibody is administered at a dose of about 1300 mg. In some embodiments, the bispecific anti-EGFR/c-Met antibody is administered at a dose of about 1350 mg. In some embodiments, the bispecific anti-EGFR/c-Met antibody is administered at a dose of about 1400 mg. In some embodiments, the bispecific anti-EGFR/c-Met antibody is administered at a dose of about 1575 mg. In some embodiments, the bispecific anti-EGFR/c-Met antibody is administered at a dose of about 1600 mg. In some embodiments, the bispecific anti-EGFR/c-Met antibody is administered at a dose of about 2100 mg. In some embodiments, the bispecific anti-EGFR/c-Met antibody is administered at a dose of about 2240 mg.

In some embodiments, the bispecific anti-EGFR/c-Met antibody is administered is administered at a dose of 1050 mg if the subject has a body weight of less than 80 kg. In some embodiments, the bispecific anti-EGFR/c-Met antibody is administered is administered at a dose of 1050 mg even if the the subject has a body weight of greater than or equal to 80 kg.

In some embodiments, the bispecific anti-EGFR/c-Met antibody is administered at a dose of 1400 mg if the subject has a body weight of greater than or equal to 80 kg.

In some embodiments, the bispecific anti-EGFR/c-Met antibody is administered once a week. In some embodiments, the bispecific anti-EGFR/c-Met antibody is administered about 1050 mg once a week. In some embodiments, the bispecific anti-EGFR/c-Met antibody is administered about 1400 mg once a week.

In some embodiments, the bispecific anti-EGFR/c-Met antibody is administered once in two weeks. In some embodiments, the bispecific anti-EGFR/c-Met antibody is administered about 1050 mg once in two weeks. In some embodiments, the bispecific anti-EGFR/c-Met antibody is administered about 1400 mg once in two weeks.

In some embodiments, the bispecific anti-EGFR/c-Met antibody is administered is administered at a dose of 1575 mg if the subject has a body weight of less than 80 kg. In some embodiments, the bispecific anti-EGFR/c-Met antibody is administered at a dose of 2100 mg if the subject has a body weight of greater than or equal to 80 kg.

In some embodiments, the bispecific anti-EGFR/c-Met antibody is administered is administered at a dose of 1600 mg if the subject has a body weight of less than 80 kg. In some embodiments, the bispecific anti-EGFR/c-Met antibody is administered at a dose of 2240 mg if the subject has a body weight of greater than or equal to 80 kg.

In some embodiments, the bispecific anti-EGFR/c-Met antibody is administered twice a week. In some embodiments, the bispecific anti-EGFR/c-Met antibody is administered once a week. In some embodiments, the bispecific anti-EGFR/c-Met antibody is administered once in two weeks. In some embodiments, the bispecific anti-EGFR/c-Met antibody is administered once in three weeks. In some embodiments, the bispecific anti-EGFR/c-Met antibody is administered once in four weeks.

In some embodiments, the bispecific anti-EGFR/c-Met antibody is administered twice a week, once a week, once in two weeks, once in three weeks or once in four weeks.

In some embodiments, the bispecific anti-EGFR/c-Met antibody is administered once a week for four weeks and once in two weeks thereafter.

Generation of Bispecific anti-EGFR/c-Met Antibodies

An exemplary bispecific anti-EGFR/c-Met antibody that can be used in the methods of the disclosures is amivantamab. Amivantamab is an IgG1 anti-EGFR/c-Met bispecific antibody described in U.S. Pat. No. 9,593,164, which is incorporated herein by reference in its entirety.

Amivantamab is a low fucose, fully human immunoglobulin G1 (IgG1)-based bispecific antibody directed against the EGFR and MET receptors, shows preclinical activity against tumors with overexpressed wild type EGFR and activation of the MET pathway. Unlike EGFR TKIs, which bind to the intracellular portion of the EGFR, amivantamab targets the extracellular domain of both EGFR and MET. Amivantamab may have at least 3 potential mechanisms of action, including 1) inhibition of ligand-dependent signaling, 2) downregulation of EGFR and MET expression levels, and 3) initiation of antibody-dependent cellular cytotoxicity (ADCC). Amivantamab is produced with low levels of fucosylation, which translates to an enhanced level of ADCC activity. The human FcyllIa receptor, critical for ADCC, binds low fucose antibodies more tightly and consequently mediates more potent and effective ADCC killing of target cancer cells (Satoh, 2006). It is hypothesized that by targeting the extracellular domain of EGFR and MET, amivantamab can inhibit receptors that display primary resistance to EGFR TKIs (Exon 20 insertion) or have acquired either EGFR resistance mutations (T790M or C797S) or secondary activation of the MET pathway (MET amplification).

Amivantamab is characterized by following amino acid sequences:

EGFR binding arm
(HCDR1, EGFR binding arm)
>SEQ ID NO: 1
TYGMH
(HCDR2, EGFR binding arm)
>SEQ ID NO: 2
VIWDDGSYKYYGDSVKG
(HCDR3, EGFR binding arm)
>SEQ ID NO: 3
DGITMVRGVMKDYFDY
(LCDR1, EGFR binding arm)
>SEQ ID NO: 4
RASQDISSALV
(LCDR2, EGFR binding arm)
>SEQ ID NO: 5
DASSLES
(LCDR3, EGFR binding arm)
>SEQ ID NO: 6
QQFNSYPLT
(HCDR1, c-Met binding arm)
>SEQ ID NO: 7
SYGIS
(HCDR2, c-Met binding arm)
>SEQ ID NO: 8
WISAYNGYTNYAQKLQG
(HCDR3, c-Met binding arm)
>SEQ ID NO: 9
DLRGTNYFDY
(LCDR1, c-Met binding arm)
>SEQ ID NO: 10
RASQGISNWLA
(LCDR2, c-Met binding arm)
>SEQ ID NO: 11
AASSLLS
(LCDR3, c-Met binding arm)
>SEQ ID NO: 12
QQANSFPIT
(VH, EGFR binding arm)
>SEQ ID NO: 13
QVQLVESGGGVVQPGRSLRLSCAASGFTFSTYGMHWVRQA
PGKGLEWVAVIWDDGSYKYYGDSVKGRFTISRDNSKNTLY
LQMNSLRAEDTAVYYCARDGITMVRGVMKDYFDYWGQGTL
VTVSS
(VL, EGFR binding arm)
>SEQ ID NO: 14
AIQLTQSPSSLSASVGDRVTITCRASQDISSALVWYQQKP
GKAPKLLIYDASSLESGVPSRFSGSESGTDFTLTISSLQP
EDFATYYCQQFNSYPLTFGGGTKVEIK
(VH, c-Met binding arm)
>SEQ ID NO: 15
QVQLVQSGAEVKKPGASVKVSCETSGYTFTSYGISWVRQA
PGHGLEWMGWISAYNGYTNYAQKLQGRVTMTTDTSTSTAY
MELRSLRSDDTAVYYCARDLRGTNYFDYWGQGTLVTVSS
(VL, c-Met binding arm)
>SEQ ID NO: 16
DIQMTQSPSSVSASVGDRVTITCRASQGISNWLAWFQHKP
GKAPKLLIYAASSLLSGVPSRFSGSGSGTDFTLTISSLQP
EDFATYYCQQANSFPITFGQGTRLEIK
HC1
>SEQ ID NO: 17
QVQLVESGGGVVQPGRSLRLSCAASGFTFSTYGMHWVRQA
PGKGLEWVAVIWDDGSYKYYGDSVKGRFTISRDNSKNTLY
LQMNSLRAEDTAVYYCARDGITMVRGVMKDYFDYWGQGTL
VTVSSASTKGPSVFPLAPSSKSTSGGTAALGCLVKDYFPE
PVTVSWNSGALTSGVHTFPAVLQSSGLYSLSSVVTVPSSS
LGTQTYICNVNHKPSNTKVDKRVEPKSCDKTHTCPPCPAP
ELLGGPSVFLFPPKPKDTLMISRTPEVTCVVVDVSHEDPE
VKFNWYVDGVEVHNAKTKPREEQYNSTYRVVSVLTVLHQD
WLNGKEYKCKVSNKALPAPIEKTISKAKGQPREPQVYTLP
PSREEMTKNQVSLTCLVKGFYPSDIAVEWESNGQPENNYK
TTPPVLDSDGSFLLYSKLTVDKSRWQQGNVFSCSVMHEAL
HNHYTQKSLSLSPGK
LC1
>SEQ ID NO: 18
AIQLTQSPSSLSASVGDRVTITCRASQDISSALVWYQQKP
GKAPKLLIYDASSLESGVPSRFSGSESGTDFTLTISSLQP
EDFATYYCQQFNSYPLTFGGGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC
HC2
>SEQ ID NO: 19
QVQLVQSGAEVKKPGASVKVSCETSGYTFTSYGISWVRQA
PGHGLEWMGWISAYNGYTNYAQKLQGRVTMTTDTSTSTAY
MELRSLRSDDTAVYYCARDLRGTNYFDYWGQGTLVTVSSA
STKGPSVFPLAPSSKSTSGGTAALGCLVKDYFPEPVTVSW
NSGALTSGVHTFPAVLQSSGLYSLSSVVTVPSSSLGTQTY
ICNVNHKPSNTKVDKRVEPKSCDKTHTCPPCPAPELLGGP
SVFLFPPKPKDTLMISRTPEVTCVVVDVSHEDPEVKFNWY
VDGVEVHNAKTKPREEQYNSTYRVVSVLTVLHQDWLNGKE
YKCKVSNKALPAPIEKTISKAKGQPREPQVYTLPPSREEM
TKNQVSLTCLVKGFYPSDIAVEWESNGQPENNYKTTPPVL
DSDGSFFLYSRLTVDKSRWQQGNVFSCSVMHEALHNHYTQ
KSLSLSPGK
LC2
>SEQ ID NO: 20
DIQMTQSPSSVSASVGDRVTITCRASQGISNWLAWFQHKP
GKAPKLLIYAASSLLSGVPSRFSGSGSGTDFTLTISSLQP
EDFATYYCQQANSFPITFGQGTRLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC

In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In some embodiments, the first domain that specifically binds EGFR comprises a heavy chain variable region (VH) of SEQ ID NO: 13 and a light chain variable region (VL) of SEQ ID NO: 14; and the second domain that specifically binds c-Met comprises the VH of SEQ ID NO: 15 and the VL of SEQ ID NO: 16.

In some embodiments, the bispecific anti-EGFR/c-Met antibody is an IgG1 isotype.

In some embodiments, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In one embodiment, the bispecific anti-EGFR/c-Met antibody comprises one or more Fc silencing mutations.

In one embodiment, the one or more Fc silencing mutations decrease affinity to Fcγ receptors.

In one embodiment, the one or more Fc silencing mutations comprise V234A/G237A/P238S/H268A/V309L/A330S/P331S.

In one embodiment, the bispecific anti-EGFR/c-Met antibody comprises a biantennary glycan structure with a fucose content between about 1% to about 15%.Antibodies with reduced fucose content can be made using different methods reported to lead to the successful expression of relatively high defucosylated antibodies bearing the biantennary complex-type of Fc oligosaccharides such as control of culture osmolality (Konno et al., Cytotechnology 64(:249-65, 2012), application of a variant CHO line Lec13 as the host cell line (Shields et al., J Biol Chem 277:26733-26740, 2002), application of a variant CHO line EB66 as the host cell line (Olivier et al., MAbs ;2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the □ 1,6-fucosyltrasferase (FUT8) gene (Mori et al., Biotechnol Bioeng88:901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem281:5032-5036, 2006, Ferrara et al., Biotechnol Bioeng 93:851-861, 2006; Xhou et al., Biotechnol Bioeng 99:652-65, 2008). In general, lowering fucose content in the glycan of the antibodies potentiates antibody-meidated cellular cytotoxicity (ADCC).

Other bispecific anti-EGFR/c-Met antibodies may also be used in the methods of the disclosure as long as they demonstrate similar characteristics when compared to amivantamab as described in U.S. Pat. No. 9,593,164. Bispecific anti-EGFR/c-Met antibodies that may be used in the methods of the disclosure may also be generated by combining EGFR binding VH/VL domains and c-Met binding VH/VL domains and testing the resulting bispecific antibodies for their characteristics as described in U.S. Pat. No. 9,593,164.

Bispecific anti-EGFR/c-Met antibodies used in the methods of the disclosure may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e. an epitope on EGFR and an epitope on c-Met. For example, the bispecific antibodies of the invention may be generated using the technology described in Int.Pat. Publ. No. WO2011/131746. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. For IgG4 antibodies, a wild-type IgG4 and a IgG4 antibody with F405L and R409K substitutions may be used. To generate bispecific antibodies, first monospecific bivalent antibody and the second monospecific bivalent antibody are engineered to have the aforementioned mutation in the Fc region, the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Bispecific anti-EGFR/c-Met antibodies used in the methods of the disclosure may also be generated using designs such as the knob-in-hole or knobs-into-holes (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody)(EMD Serono), and the Biclonic (Merus).

In the “knob-in-hole” strategy (see, e.g., Intl. Publ. No. WO 2006/028936) select amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

CrossMAb technology, in addition to utilizing the “knob-in-hole” strategy to promoter Fab arm exchange utilizes CH1/CL domain swaps in one half arm to ensure correct light chain pairing of the resulting bispecific antibody (see e.g. U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by following substitutions (expressed as modified positions in the first CH3 domain of the first heavy chain/modified position in the second CH3 domain of the second heavy chain): L351Y_F405A_Y407V/T394W, T366I K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V T366L_K392L_T394W as described in U.S. Patent Publ. No. US2012/0149876 or U.S. Patent Publ. No. US2013/0195849.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

### Exemplary Embodiments

1. A method of treating liver cancer in a subject in need thereof, comprising administering to the subject a therapeutically effective amount of a bispecific anti-epidermal growth factor receptor (EGFR)/hepatocyte growth factor receptor (c-Met) antibody.
2. The method of embodiment 1, wherein the liver cancer is hepatic cellular carcinoma (HCC).
3. The method of embodiment 1 or 2, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.
4. The method of embodiment 3, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.
5. The method of embodiment 3 or 4, wherein the bispecific anti-EGFR/c-Met antibody is an IgG1 isotype.
6. The method of any one of embodiments 1-5, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.
7. The method of any one of embodiments 1-6, wherein the bispecific anti-EGFR/c-Met antibody comprises a biantennary glycan structure with a fucose content of about between 1% to about 15%.
8. The method of any one of embodiments 1-7, wherein the bispecific anti-EGFR/c-Met antibody is administered intravenously to the subject.
9. The method of embodiment 8, wherein the bispecific anti-EGFR/c-Met antibody is administered at a dose of between about 350 mg to about 1400 mg.
10. The method of embodiment 9, wherein the bispecific anti-EGFR/c-Met antibody is administered at a dose of about 350 mg, 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg or 1400 mg.
11. The method of embodiment 10, wherein the bispecific anti-EGFR/c-Met antibody is administered at a dose of 1050 mg.
12. The method of embodiment 10, wherein the bispecific anti-EGFR/c-Met antibody is administered at a dose of 1400 mg.
13. The method of any one of embodiments 1-7, wherein the bispecific anti-EGFR/c-Met antibody is administered subcutaneously or intradermally to the subject.
14. The method of embodiment 13, wherein the bispecific anti-EGFR/c-Met antibody is administered subcutaneously or intradermally at a dose sufficient to achieve a therapeutic effect in the subject.
15. The method of any one of embodiments 1-14, wherein the bispecific anti-EGFR/c-Met antibody is administered twice a week, once a week, once in two weeks, once in three weeks or once in four weeks.
16. The method of any one of embodiments 1-15, wherein the bispecific anti-EGFR/c-Met antibody is administered twice a week, once a week, once in two weeks, once in three weeks or once in four weeks.
17. The method of embodiment 16, wherein the bispecific anti-EGFR/c-Met antibody is administered once a week for four weeks and once in two weeks thereafter.
18. The method of any one of embodiments 1-17, wherein the subject has received prior treatment.
19. The method of embodiment 18, wherein the prior treatment comprises a multi-targeted kinase inhibitor (MKI), an immunotherapy, anti-VEGF/VEGFR therapy, or a combination thereof
20. The method of embodiment 19, wherein the multi-targeted kinase inhibitor (MKI) is sorafenib regorafenib, lenvatinib, cabozantinib, apatinib, or a combination thereof
21. The method of embodiment 19, wherein the immunotherapy comprises a PD-(L)1 axis inhibitor, or a CTLA-4 inhibitor.
22. The method of embodiment 22, wherein the PD-(L)1 axis inhibitor comprises atezolizumab, nivolumab, pembrolizumab, camrelizumab, tislelizumab, or a combination thereof.
23. The method of embodiment 22, wherein the CTLA-4 inhibitor comprises ipilimumab.
24. The method of embodiment 19, wherein the anti-VEGF/VEGFR therapy comprises bevacizumab or ramucirumab.
25. The method of any one of embodiments 1-17, wherein the subject is treatment naïve.
26. The method of embodiments 18 or 19, wherein the prior treatment comprises a multi-targeted kinase inhibitor (MKI).

### EXAMPLES

The following examples are provided to further describe some of the embodiments disclosed herein. The examples are intended to illustrate, not to limit, the disclosed embodiments.

### Example 1. A Phase 2, Open-Label Study to Evaluate the Safety, Efficacy and Pharmacokinetics of Amivantamab Monotherapy in Participants with Previously Treated Advanced Hepatocellular Carcinoma (HCC)

### Study Rationale

HCC has been known to highly express EGFR and MET and the expression of these proteins correlate with poor prognosis. Although many agents targeting EGFR and MET are parts of standard of care for many tumor types, no anti-EGFR therapy and MET inhibitors have been approved in HCC. One potential mechanism behind the lack of clinical activities is the redundancy of receptor tyrosine kinases (RTKs), including EGFR and MET, in upregulating downstream oncogenic signaling (Saraon 2021). Thus, inhibiting EGFR or MET alone may not be sufficient in eliciting clinical activity. In addition, the lack of consensus and reliable biomarker strategies to select participants may also contribute to the failure of many clinical studies. As a bispecific antibody capable of engaging the extracellular domains of both EGFR and MET receptors, amivantamab has a unique mechanism of action that suggests it has the potential to control EGFR overexpressing and/or MET-overexpressing tumors in HCC patients. In addition to the known Fc-independent EGFR and MET signal inhibition, multiple Fc-dependent mechanisms (including antibody-dependent cellular cytotoxicity and macrophage- and monocyte-mediated trogocytosis) have been implicated in the robust anti-tumor effect of amivantamab (Moores 2016; Grugan 2017; Vijayaraghavan 2020). The complex immune microenvironment in HCC has provided opportunities for many immunotherapies. The Fc-driven mechanisms of amivantamab may mobilize immune cells in HCC tumor microenvironment to contribute to its clinical benefit.

In a panel of EGFR and MET wild-type HCC patient-derived xenograft (PDX) models, amivantamab demonstrated significant anti-tumor activity in 4 out of 9 tumor models tested. In one EGFR and MET highly expressed PDX model, amivantamab induced full tumor regression and the activity is durable for months following dosing withdraw. As a comparison, amivantamab showed better tumor growth inhibition (TGI) than the SOC treatment sorafenib and capmatinib, a MET inhibitor under development for HCC. The activity correlated with the amivantamab MOA including EGFR/MET signaling inhibition, receptor downmodulation and Fc-dependent immune cells contribution. See Example 2.

Furthermore, preliminary biomarker evaluation has suggested amivantamab to be more active in patients with higher expression of EGFR and MET in osimertinib-relapsed EGFR mutated NSCLC disease (Bauml ASCO 2021).

This study aims to evaluate the clinical activity of amivantamab as a monotherapy in HCC participants who had received at least 1 prior lines of standard therapy. A safety evaluation will be conducted in 6 participants to evaluate the safety of amivantamab in HCC participants. Approximately 60 participants [at least 30 participants with first line Immuno-Oncology (I/O) combination treatment] will be enrolled in the study. A retrospective analysis will be conducted to understand the correlation of biomarker(s), including the expression level of receptor, ligand(s) in the EGFR pathway and/or MET pathway, or other biomarkers, with responsive population.

### Objectives and Endpoints

### Primary Objective

The primary objective for this study is to characterize the preliminary antitumor activity of amivantamab at the recommended phase 2 dose (RP2D) in participants with previously systemically treated HCC on the basis of the following endpoint(s):
Objective response rate (ORR) as determined by investigator, according to the Response Criteria in Solid Tumors (RECIST) v1.1

### Secondary Objectives

A secondary objective for this study is to assess additional measures of clinical benefit with amivantamab on the basis of the following endpoints:
Duration of response (DOR)
Disease control rate (DCR)
Progression-free survival (PFS)
Overall survival (OS)

Another secondary objective for this study is to characterize the safety and tolerability of amivantamab in participants with advanced HCC on the basis of the following endpoint(s):
Adverse events defined by the National Cancer Institute Common Terminology Criteria for Adverse Events (NCI CTCAE) Criteria version 5.0

Another secondary objective for this study is to assess the pharmacokinetics (PK) and immunogenicity of amivantamab following multiple intravenous dose administrations on the basis of the following endpoint(s):
Serum PK parameters including but not limited to maximum serum concentration (Cmax), time to reach the maximum serum concentration (Tmax), area under the curve (AUC)(t1−t2), AUCtau, serum concentration immediately prior the next study treatment administration (Ctrough), and accumulation ratio; incidence of antidrug antibodies Exploratory Objectives

An exploratory objective for this study is to further explore the clinical benefit achieved with amivantamab treatment through alternative response criteria on the basis of the following endpoint(s):
ORR, as determined by Investigator Review, according to mRECIST

Additional objectives for this study are to explore tissue biomarkers (including but not limited to EGFR, MET expression) and blood biomarkers predictive of clinical response and resistance to amivantamab; explore the relationship between serum PK, pharmacodynamic (PD) markers and clinical outcome; explore the relationship of etiology (e.g., HepB, HepC) and clinical response; explore primary and acquired resistance to amivantmab treatment.

### Study Design

### Overview of Study Design

This is an open-label, multicenter Phase 2 interventional study in participants with previously treated advanced HCC.

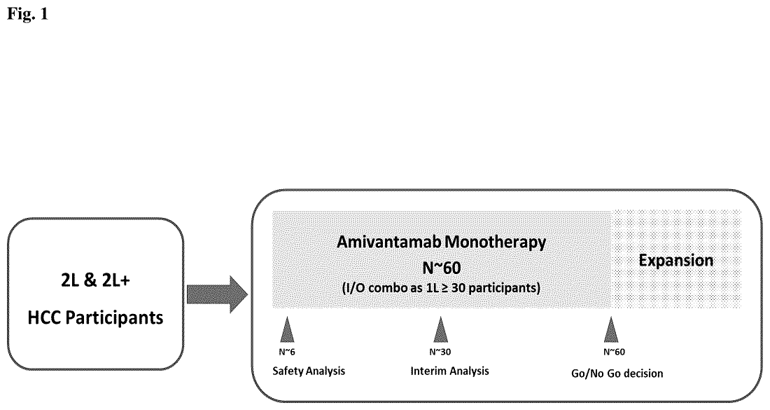

Participants will receive amivantamab treatment at the RP2D, as shown in FIG. 1. An early review of safety will be performed after 6 particiapnts have been enrolled and have completed at least 1 complete cycle of amivantamab therapy to confirm the consistent safety profile.

In total, approximately 60 participants (at least 30 participants with first line I/O combination treatment) will be enrolled in the study. Two interim analyses are planned: an early review of safety after the first 6 participants have completed at least 1 cycle of amivantamab therapy and an early review of efficacy after 30 participants have been enrolled and are response evaluable. An expansion phase may be opened to confirm the clinical response in all-comer population based on the efficacy data from the 60 participants. A retrospective analysis will be conducted to understand the correlation of biomarker(s), including the expression level of receptor, ligand(s) in the EGFR pathway and/or MET pathway, or other biomarkers, with responsive population.

The study will include a screening phase, a treatment phase, and a follow-up phase.

Sequence and duration of study phases/periods:
Screening: Day-28 to Day-1, beginning with the signing of the informed consent form (ICF). Treatment: 28-Day Cycles, from Day 1 until End of Treatment visit, approximately 30 days after discontinuation of study treatment due to disease progression, death, or discontinuation (e.g., withdrawal of consent, adverse event)

Follow-up: every 12 weeks from the last dose of study treatment or disease progression (whichever comes first), until death, lost to follow-up, or withdrawal of consent, whichever comes first.

End of study definition (per EU Directive): the end of study will occur after all participants have discontinued therapy with study treatment and have at least 6 months of follow-up or have discontinued from the study.

Key study measures:
Efficacy: disease assessments, per RECIST v1.1;
Safety: adverse events per CTCAE v5.0, clinical safety laboratory assessments, vital signs

### Study Population

### Inclusion Criteria

Participant sex (biologically): Both male and female.

Participant age: Minimum age: 18 years of age (or the legal age of consent in the jurisdiction in which the study is taking place).

Participants have histologically or cytologically confirmed diagnosis of HCC (fibrolamellar and mixed hepatocellular/cholangiocarcinoma subtypes are not eligible) based on pathology report, who have BCLC Stage C disease or BCLC Stage B disease not amenable to locoregional therapy or refractory to locoregional therapy, and not amenable to a curative treatment approach.

Participants have progressed on or after, been intolerant or refused to at least 1 prior line of systemic therapy. Prior therapies may include multi-targeted kinase inhibitor (MKI) and/or immunotherapy (eg, PD 1/L1-containing therapy).
Progression within 24 weeks of prior adjuvant or neoadjuvant therapy will be considered as 1 prior line of systemic therapy for the purpose of meeting the eligibility criteria. Participant should have documented progression on or after the last line of therapy.
The intolerance or refusal of further therapy should be documented in medical record.
Note: A line of therapy is defined as a treatment regimen with subsequent disease progression.

Cirrhotic status of Child-Pugh Class A (A5 or A6) within 7 days of first dose of study.

Participants have a predicted life expectancy of greater than 3 months.

Participants have measurable disease according to RECIST Version 1.1. The selected target lesions must meet 1 of 2 criteria: a) not previously treated with local therapy or b) within the field of prior local therapy but with documented subsequent progression as per RECIST v1.1. If only 1 measurable lesion exists, it may be used for the screening biopsy if the baseline tumor assessment scans are performed ≥7 days after the biopsy. Note: The same image acquisition and processing parameters should be used throughout the study for a given participant.

Participants have Eastern Cooperative Oncology Group (ECOG) performance status 0 or 1 within 7 days of first dose of study drug.

Participants are eligible to enroll if they have non-viral-HCC or if they have HBV-, or HCV-HCC, defined as follows: (a) chronic HBV infection as evidenced by detectable HBV surface antigen or HBV DNA. Participants with chronic HBV infection are on antiviral therapy [per local standard of care, eg, entecavir (ETV) or tenofovir disoproxil fumarate (TDF)] for a minimum of 14 days prior to study entry and willingness to continue treatment for the length of the study, and HBV viral load is less than 2000 IU/mL prior to first dose of study drug. Those participants who are anti-HBc (+), and negative for HBsAg, and negative for anti-HBs, and have an HBV viral load under 20 IU/mL do not require HBV anti-viral prophylaxis, but need close monitoring; (b) active or resolved HCV infection as evidenced by detectable HCV RNA or antibody.

Participants have adequate organ and bone marrow function as follows, without history of red blood cell transfusion, platelet transfusion or albumin infusion within 14 days prior to the date of the laboratory test.
Adequate hematologic function:
Hemoglobin ≥9 g/dL
Absolute neutrophil count ≥1500/mm3, without use of granulocyte colony stimulating factor (G-CSF) within 10 days prior to the date of the test
Platelets ≥75×103/μL
For participants not receiving therapeutic anticoagulation: international normalized ratio (INR) or activated partial thromboplastin time (aPTT)≤2× ULN (upper limits of normal)
Adequate hepatic function with serum albumin ≥3.1g/dL, serum bilirubin≤2.5× ULN, and aspartate aminotransferase (AST) and alanine aminotransferase (ALT)≤5× ULN
Adequate renal function with ≤creatinine≤1.5× ULN or creatinine clearance>50 mL/min for participant with creatinine levels >1.5× institutional ULN (Cockcroft-Gault formula)

### Exclusion Criteria

Participant has an uncontrolled illness, including but not limited to the following: a) uncontrolled diabetes; b) ongoing or active bacterial infection (includes infection requiring treatment with antimicrobial therapy [participants will be required to complete antibiotics 1 week before enrollment]), symptomatic viral infection, or any other clinically significant infection; c) active bleeding diathesis; d) psychiatric illness/social situation that would limit compliance with study requirements; e) impaired oxygenation requiring supplemental oxygen.

Target Disease Exceptions: a) known fibrolamellar HCC, sarcomatoid HCC, or mixed cholangiocarcinoma and HCC; b) prior liver transplant; c) history of hepatic encephalopathy; d) portal vein invasion at the main portal branch (Vp4), inferior vena cava, or cardiac involvement of HCC based on imaging; e) any current moderate or severe ascites as measured by physical examination and that requires active paracentesis for control; f) brain metastases, history of leptomeningeal disease or spinal cord compression.

Participant had prior approved therapy may include MKI and/or PD-1/L1-containing therapy within 2 weeks or 4 half-lives whichever is longer. For agents with long half-lives, the maximum required time since last dose is 28 days. Toxicities from previous anticancer therapies should have resolved to baseline levels or to Grade 1 or less, (except for alopecia or Grade≤2 peripheral neuropathy or Grade≤2 hypothyroidism stable on hormone replacement).

Participant had surgery, radiotherapy, and/or locoregional therapy within 4 weeks before the first administration of study treatment. Minor surgery (e.g., simple excision, tooth extraction) must have occurred at least 7 days prior to the first dose of trial treatment (Cycle 1, Day 1). Participant must have recovered adequately (i.e., Grade≤1 or baseline, except for post-radiation skin changes [any grade]) from the toxicity and/or complications from any intervention prior to starting therapy.

Participant has received prior EGFR or MET-directed therapies.

Evidence of portal hypertension with bleeding esophageal or gastric varices within the past 3 months.

Participant has a history of (non-infectious) interstitial lung disease (ILD)/pneumonitis that required steroids, or has current ILD/pneumonitis, or where suspected ILD/pneumonitis cannot be ruled out by imaging at screening.

Participant has an active malignancy (i.e., progressing or requiring treatment change in the last 12 months) other than the disease being treated under study. The only allowed exceptions are: a) non-muscle invasive bladder cancer treated within the last 24 months that is considered completely cured; b) skin cancer (non-melanoma or melanoma) treated within the last 24 months that is considered completely cured; c) non-invasive cervical cancer treated within the last 24 months that is considered completely cured; d) localized prostate cancer (NOM0): with a Gleason score of 6, treated within the last 24 months or untreated and under surveillance, with a Gleason score of 3+4 that has been treated more than 6 months prior to full study screening and considered to have a very low risk of recurrence, or history of localized prostate cancer and receiving androgen deprivation therapy and considered to have a very low risk of recurrence; e) breast cancer: adequately treated lobular carcinoma in situ or ductal carcinoma in situ, or history of localized breast cancer and receiving antihormonal agents and considered to have a very low risk of recurrence; f) malignancy that is considered cured with minimal risk of recurrence.

Participant has a history of clinically significant cardiovascular disease including, but not limited to: a) diagnosis of deep vein thrombosis or pulmonary embolism within 4 weeks prior to the first dose of study treatment or any of the following within 6 months prior to the first dose of study treatment: myocardial infarction, unstable angina, stroke, transient ischemic attack, coronary/peripheral artery bypass graft, or any acute coronary syndrome. Clinically non-significant thrombosis, such as non-obstructive catheter-associated clots, are not exclusionary; b) prolonged corrected QT interval using Fridericia's formula (QTcF) >480 msec or clinically significant cardiac arrhythmia or electrophysiologic disease (eg, placement of implantable cardioverter defibrillator or atrial fibrillation with uncontrolled rate). Note: Participants with cardiac pacemakers who are clinically stable are eligible; c) uncontrolled (persistent) hypertension: systolic blood pressure >160 mm Hg; diastolic blood pressure >100 mm Hg Congestive heart failure (CHF) defined as New York Heart Association (NYHA) class III IV or Hospitalization for CHF (any NYHA class) within 6 months of study enrollment; d) pericarditis/clinically significant pericardial effusion; e) myocarditis.

Participant has known allergies, hypersensitivity, or intolerance to excipients of amivantamab.

Participant has received a live or live attenuated vaccine within 3 months before randomization.

Participant has, or will have, any of the following: a) an invasive operative procedure (except the treatment for the ascites should be discussed with sponsor's medical monitor) with entry into a body cavity, within 4 weeks or without complete recovery before C1D1. If needed, abdominal biopsy for baseline tumor tissue sample may be done less than 4 weeks prior to C1D1, as long as the participant has adequately recovered from the procedure prior to the first dose of study treatment in the clinical judgement of the investigator; b) significant traumatic injury within 3 weeks before the start of C1D1 (all wounds must be fully healed prior to Day 1); c) expected major surgery while the investigational agent is being administered or within 6 months after the last dose of study treatment.

Active co-infection with: a) both hepatitis B and C as evidenced by detectable HBV surface antigen or HBV DNA and HCV RNA. (Participants with a history of HCV infection but who are negative for HCV RNA by polymerase chain reaction (PCR) will be considered non-infected with HCV.); b) hepatitis D infection as evidenced by hepatitis D virus (HDV) antibody in participants with hepatitis B.

Participant is known to be positive for human immunodeficiency virus (HIV), with 1 or more of the following: a) not receiving highly active antiretroviral therapy (ART); b) had a change in ART within 6 months of the start of screening; c) receiving ART that may interfere with study treatment; d) CD4 count<350 at screening; e) acquired immunodeficiency syndrome (AIDS)-defining opportunistic infection within 6 months of start of screening; f) not agreeing to start ART and be on ART >4 weeks plus having HIV viral load <400 copies/mL at end of 4-week period (to ensure ART is tolerated and HIV controlled).

### Study Intervention(s)

Group/Arm Name: Group/Arm A
Intervention Name (Drug): Amivantamab
Dose Formulation: 7 mL vial
Unit Dose Strength(s): 350 mg/vial with concentration of 50 mg/mL; iv infusion will be prepared at the site in ˜250 mL diluent
Dosage Level(s) and Frequency:
   Cycle 1: Once weekly (with the first dose split over Day 1 [350 mg] and Day 2 [700 mg if body weight is<80 kg or 1,050 mg if body weight is ≥80 kg]);
   Cycles 2+: Days 1 and 15 of each cycle.
Route of Administration: IV infusion
Dosing instructions: per Investigational Product Preparation and Administration Instructions.
Use: Experimental.
Investigational Medicinal Product (IMP): Yes.
Non-Investigational Medicinal Product (NMP): No

### Dose Modification(s)

The following guidance is followed for dose delay and modification of the amivantamab based on the toxicity grade of adverse events other than rash, infusion-related reactions, liver chemistry abnormalities, and pulmonary toxicity.

Guidance for Amivantamab Dose Delay and Modification for Toxicities (Other Than Rash, Infusion-Related Reaction, Liver Toxicity, or Pulmonary Toxicity) is shown in Table 1.

TABLE 1
Guidance for Amivantamab Dose Delay and Modification for Toxicities

| Toxicity | | Length of | Dose Modification of Amivantamab after |
|---|---|---|---|
| Grade^{a} | Action^{b} | Interruption^{d} | Resolution of Adverse Event^{c} |
| 1 | None | N/A | Continue at current dose level. Consider |
| | | | supportive care according to local standards |
| | | | as appropriate. |
| 2 | None or | ≤7 days | If interrupted, restart at current dose level. |
| | consider | >7 days | If interrupted, consider restart at next lower |
| | interruption | | dose level. |
| 3 | Interrupt | ≤7 days | Restart at current dose level. |
| | amivantamab | >7 days | Restart at next lower dose level. |
| 4 | Interrupt | ≤7 days | Restart at next lower dose level |
| | amivantamab | >7 days | Consider permanently discontinuing |
| | | | amivantamab. Participants considered by |
| | | | the investigator and sponsor to be benefiting |
| | | | from treatment may be continued at a lower |
| | | | dose upon satisfactory resolution of the toxicity. |

^{a}Per National Cancer Institute - CTCAE version 5.0.
^{b}For all toxicities, consider supportive care according to protocol or local standards (if no protocol guidance provided), as appropriate.
^{c}Resolution defined as: Grade ≤ 1 non-hematologic toxicity or back to baseline.
^{d}If interruption occurs for more than 1 cycle, contact the Medical Monitor to discuss retreatment.

Guidance for stepwise dose modification of amivantamab is outlined in Table 2.

TABLE 2
Dose Reduction for Amivantamab:

| | Amivantamab | |
|---|---|---|
| Dosage Level | Participant < 80 kg | Participant ≥ 80 kg |
| 1 (initial dosage) | 1050 mg Q2W | 1400 mg Q2W |
| 2 | 700 mg Q2W | 1050 mg Q2W |
| 3 | 700 mg Q2W | 1050 mg Q2W |
| 4 | 350 mg Q2W | 700 mg Q2W |
| 5 | Withhold | Withhold |

Q2W = every 2 weeks (eg, Day 1 and Day 15 of each 28-day cycle).

### Statistical Considerations

### Statistical Hypotheses

The hypothesis is that amivantamab monotherapy will lead to objective response rate (ORR) higher than 10% (ie, H₀: ORR≤10%) in participants with advanced HCC.

### Sample Size

Assume the ORR is 20% for the entire population, approximately sixty participants with advanced HCC will be enrolled to have over 85% probability to observe the ORR greater than 15%. With this rule, false positive rate could be controlled under 15% if the true ORR is less than 10%.

Additional participants (i.e., approximately 40) may be enrolled in an expansion phase to test the ORR of amivantamab against the null hypothesis with alpha=0.05 (two-sided) if supported by emerging data.

Anti-tumor activity within biomarker-defined subpopulations will be investigated to identify the most responsive patient population who may benefit from amivantamab. With the prevalence of 50% of the entire population having 25% ORR, it is expected to have approximately 90% power to observe the posterior probability (ORR>15%)≥50% in this population. On the other hand, if the biomarker defined population has undesirable ORR (ie, 10%), there is less than 20% probability to observe the posterior probability (ORR>15%)≥50%. More examples are given in Table 3.

TABLE 3

| | | Power to observe |
|---|---|---|
| Prevalence | ORR for | posterior probability |
| of effective | effective | (ORR > 15%) ≥ 50% |
| population | population | in effective population |
| 25% | 10% | 18% |
| | | (type I error) |
| | 20% | 60% |
| | 25% | 76% |
| | 30% | 87% |
| 50% | 10% | 18% |
| | | (type I error) |
| | 20% | 74% |
| | 25% | 90% |
| | 30% | 97% |

### Statistical Methods

The statistical analysis plan will be finalized prior to database lock, and it will include a more technical and detailed description of the statistical analyses described in this section. This section is a summary of the planned statistical analyses of the most important endpoints including primary and key secondary endpoints.

All continuous variables will be summarized using number of participants (n), mean, standard deviation (SD), median, minimum and maximum. Discrete variables will be summarized with number and percent. The Kaplan-Meier product limit method will be used to estimate the time-to-event variables including median survival time.

### Example 2. In Vitro and In Vivo Studies to Support Amivantamab in Hepatic Cellular Carcinoma

The objective of this Example was to characterize the in vitro and in vivo efficacy of amivantamab in HCC. This Examples reviews data on inhibition of receptor phosphorylation, inhibition of cell viability and inhibition of xenograft tumor growth in vivo.

### Materials and Methods

Cell lines The cell origin and genomic profile of each cell line is shown in Table 4.

TABLE 4
HCC Cell Line Profiling

| Cell line | Origin | EGFR GCN* | CMet GCN* | HGF expression | pMet level |
|---|---|---|---|---|---|
| NCI-H292 | Lung | 2 | 2 | − | ND |
| IM95 | Gastric cancer | 3 | 3 | +++ | ND |
| MKN45 | Gastric | NA | AMP | − | ND |
| MHCC97H | HCC | NA | NA | − | High |
| HCCLM3 | HCC | NA | NA | − | High |
| JHH4 | HCC | 2 | 4 | + | ND |
| JHH5 | HCC | 2 | 3 | ++ | ND |
| SNU398 | HCC | 2 | 2 | +++ | ND |

Note:
WT: wild-type;
AMP: amplification
NA: not available;
ND: not detected;
GCN: Gene Copy Number
*data from CCLE (Cancer Cell Line Encyclopedia)
HGF expression: +++: highest mRNA level; ++: high expression, +: positive expression; −: low expression

### Animals

6-8 weeks old female Balb/c nude mice were used in the study. All the procedures related to animal handling, care, and the treatment in this study were performed according to guidelines approved by the Institutional Animal Care and Use Committee (IACUC) following the guidance of the Association for Assessment and Accreditation of Laboratory Animal Care (AAALAC). At the time of routine monitoring, the animals were checked for any effects of tumor growth on normal behavior such as mobility, food and water consumption, body weight gain/loss, eye/hair matting and any other abnormal effect. Death and observed clinical signs were recorded on the basis of the numbers of animals within each subset.

### CellTiter-Glo Luminescent Cell Viability Assay

Each cell line was treated with amivantamab in the absence and presence of one concentration of HGF in both 96-well tissue culture plate and 96-well ultra-low attach surface plate. Vehicles of test articles and culture medium were also included. Amivantamab concentrations started at 100 μM then diluted in 3.16 fold with a total of 9 points.

The data was displayed graphically using GraphPad Prism 5.0. In order to calculate ICso, a dose-responsive curve was fitted using nonlinear regression model with a sigmoidal dose response. The formula of surviving rate is shown below, and the IC₅₀ was automatically produced by GraphPad Prism 5.0. The surviving rate (%)=(LumTest article−LumMedium control)/(LumNone treated−LumMedium control)×100%.

### 3D Soft Agar Colony Formation Assay

A 1% (1 g/100 mL) agarose (LMP Agarose) solution was prepared and mixed with equal volumes of 2× complete growth medium giving a final concentration of 0.5% agarose.

One volume of 3.5% agarose and four volumes of 1× complete growth medium were mixed giving a final concentration of 0.7% agarose. Equal volumes of 0.7% agarose and cell suspension with drugs were mixed. Amivantamab concentrations started at 31.6 μM then diluted in 3.16 fold with a total 5 points.

Cultures were monitored using a microscope, until colonies of preferred cell number were achieved (6-9 days). 80 μL CellTiter-Glo was added to each well. The plates were incubated 90 minutes to induce cell lysis at room temperature then luminescence was recorded. Patient-derived HCC xenograft model establishment

Patient-derived HCC xenograft models were generated using fresh tumor tissue from HCC patients. Briefly, surgically excised HCC tissue was cut into approximately 15 mm³ segments and implanted into immune-deficient mice subcutaneously. Subsequent passages were made into nude mice once the grafted tumors grew up and reached a size of ˜500 mm³, In vivo anti-tumor efficacy studies

When the xenograft tumor volumes reached 150-250 mm³, the tumor-bearing mice were randomized into groups of 8-10 mice, and treated with either control vehicle or amivantamab at 10 mg/kg twice weekly or JNJ-38877618 (CAS Registry No. 943540-74-7) at 50 mg/kg once daily. Xenograft tumor volume and body weight of the tumor-bearing mice were measured twice weekly. Subcutaneous tumor volumes (V) calculated by the formula: V=(length x width²)/2. Percentage of tumor growth inhibition (%TGI) was calculated as the formula: TGI%=[1−(change of tumor volume in treatment group/change of tumor volume in control group)]×100 and was used for the evaluation of anti-tumor efficacy. The xenograft tissues were collected at different time points post last dose and used for western blot to detect the downstream signal pathway modulation upon compound treatment.

### Statistical Analysis

The differences between the mean values of tumor size for comparing groups were analyzed for significance by independent t test using GraphPad Prism software. P<0.05 was considered to be statistically significant.

### Results

Expression Level of pMet and EGFR in HCC Cell Lines

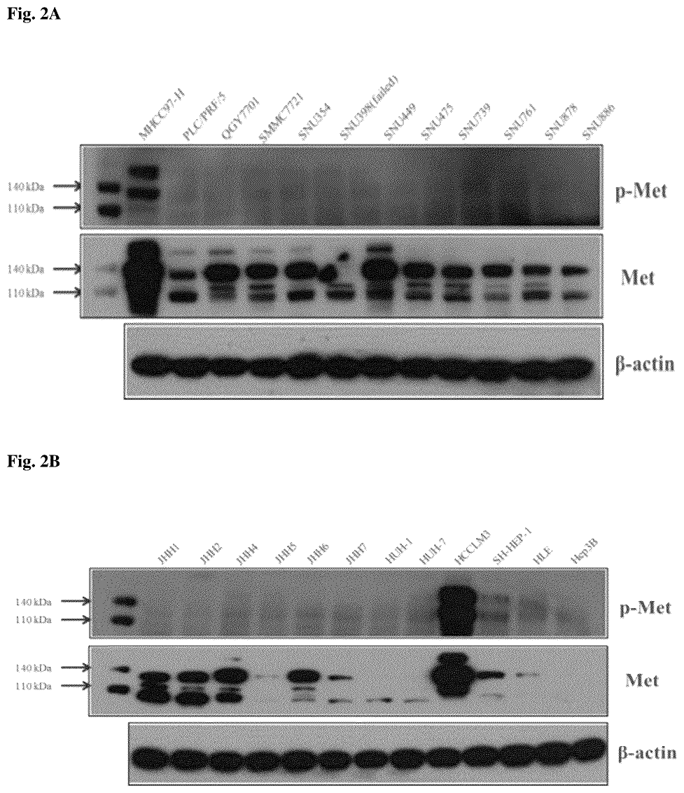

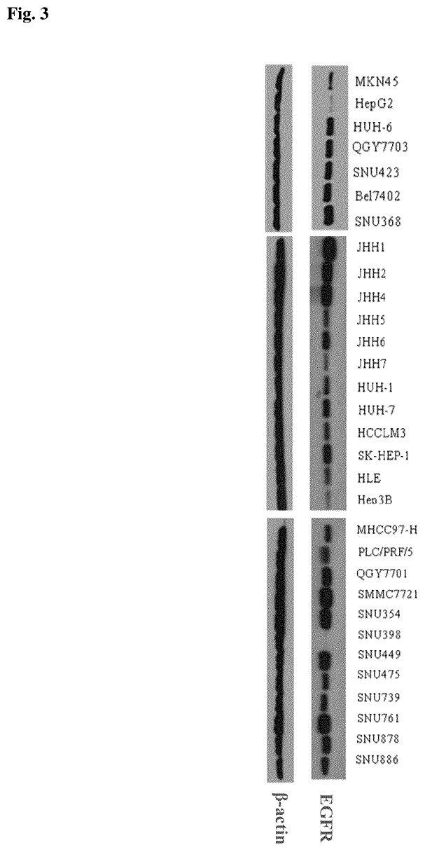

The pMet, total Met and EGFR expression level in HCC cell lines were measured by western blot. As shown in FIGS. 2A and 2B, only two HCC cell lines, MIFICC97H and HCCLM3, showed high pMet and total Met expression levels. pMet expression level was undetected in other HCC cell lines, and the expression level of total Met was also low in Huh-1, Huh-7 and Hep3B cell lines. On the other hand, EGFR expression was detected in most HCC cell lines tested except in SNU398 (FIG. 3). Combined with the genetic profiling data from Cancer Cell Line Encyclopedia (CCLE, Table 4), cell lines were selected for further studies.

### In Vitro HCC Cell Growth Inhibition by Amivantamab

To study the effect of amivantamab in HCC cell line proliferation, CellTiter-Glo luminescent cell viability assay was employed. The genetic profiling of the cells selected for this analysis is listed in Table 4. Among these cells, NCI-H292 is wild type without MET amplification or HGF high expression, MIFICC97H and HCCLM3 display pMet overexpression, IM95 and SNU398 have high HGF expression and MKN45 has MET amplification.

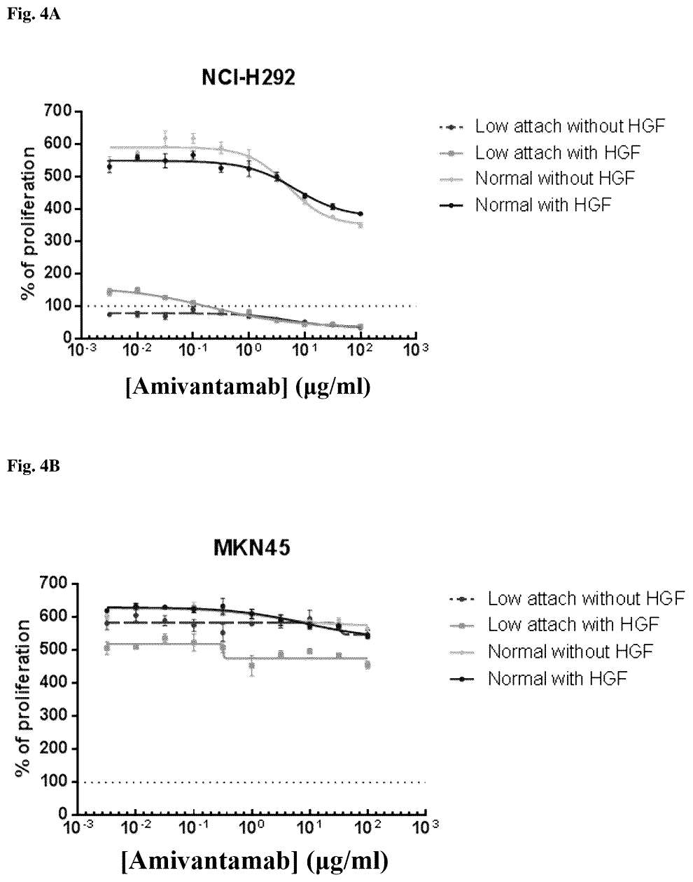

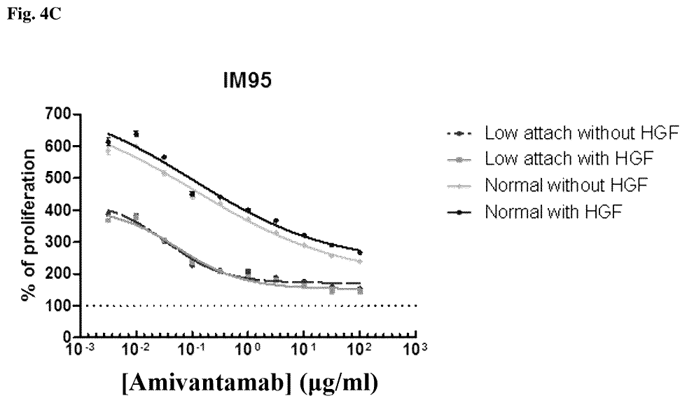

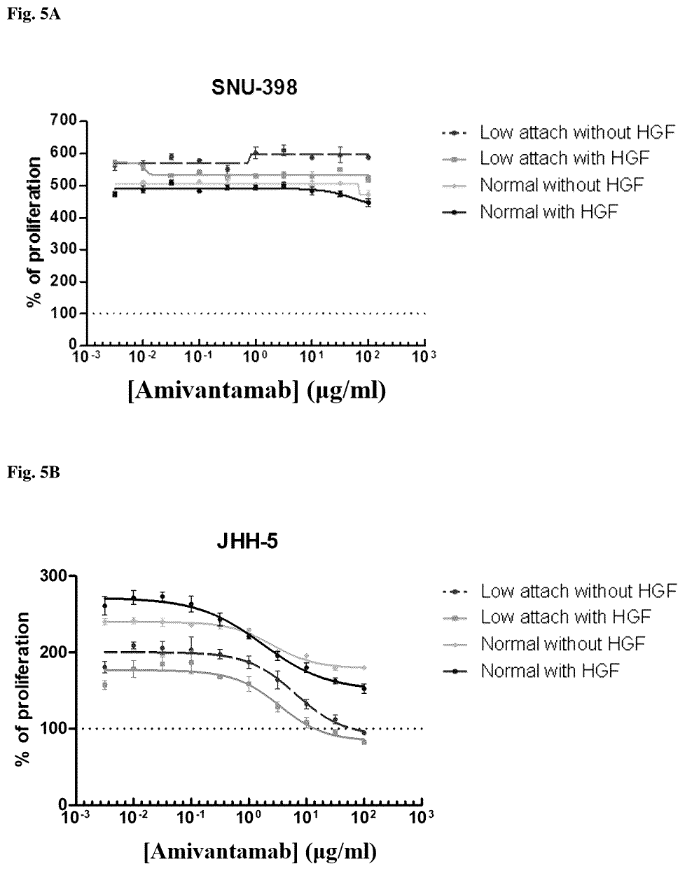

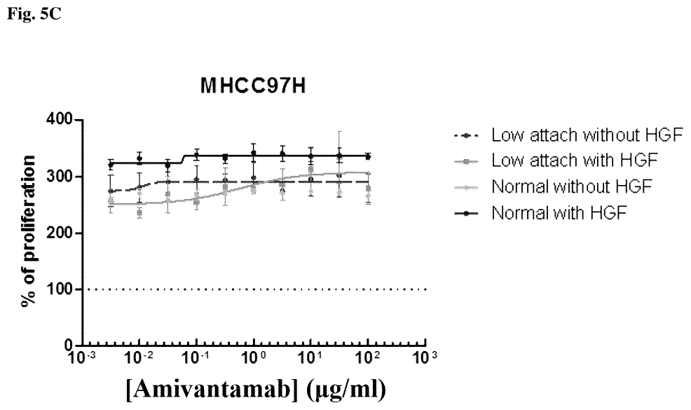

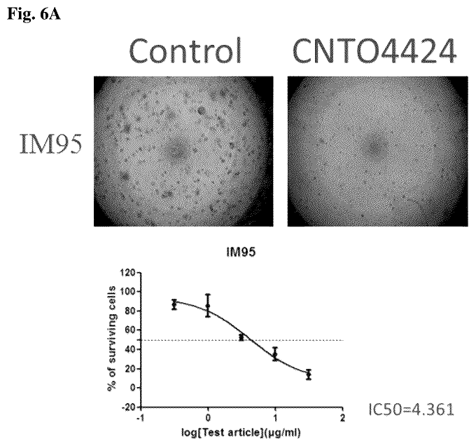

As shown in Table 5, amivantamab significantly inhibited cell growth of IM95 cell line with high HGF expression, but had no effect on other cell lines tested, including the cell line with MET amplification, MKN45, the cell lines with pMet overexpression, MIFICC97H and HCCLM3, and the cell lines with HGF high expression, SNU398 and JHHS (FIGS. 4A-4C and FIGS. 5A-5C). It was demonstrated that low attach surface plate and the implement of HGF can sensitize IM95 and NCI-H292 cells to Amivantamab (FIGS. 4A-4C).

TABLE 5
IC₅₀ (μg/ml) of Amivantamab against HCC cell lines

| Cell Line | Group1 | Group2 | Group3 | Group4 |
|---|---|---|---|---|
| H292 | 58.2 | 0.9 | NA | NA |
| MKN45 | NA | NA | NA | NA |
| IM95 | 0.57 | 0.81 | 8.57 | 16.12 |
| SNU398 | NA | NA | NA | NA |
| JHH5 | NA | NA | NA | NA |
| MHCC97H | NA | NA | NA | NA |
| JHH4 | NA | NA | NA | NA |
| HCCLM3 | NA | NA | NA | NA |

IC₅₀ values are calculated using GraphPad Prism software.
NA = Not Available
Group 1: low attach plate without HGF;
Group 2: low attach plate with HGF;
Group 3: normal plate without HGF;
Group 4: normal plate with HGF

### Effect of Amivantamab on HCC Colony Formation

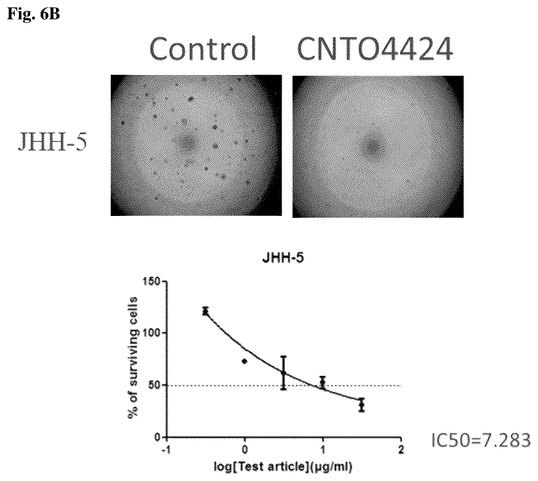

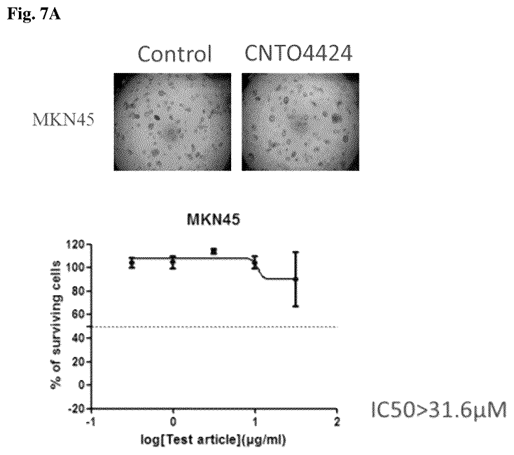

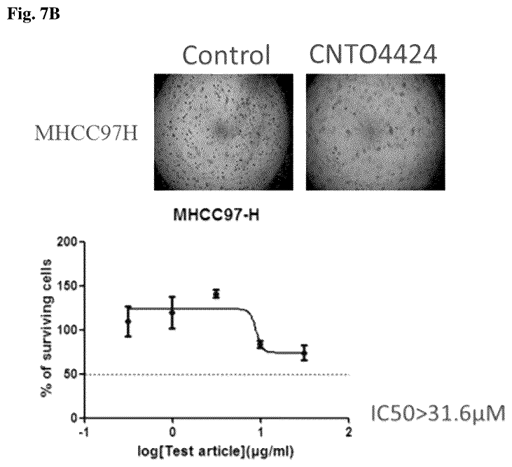

The activity of antibodies has been shown to be limited on 2D cell proliferation assay. To circumvent this problem and to further study the effect of Amivantamab on HCC cell line proliferation, a 3D soft agar assay was used which reflects the ability of anchorage-independent growth. Cell lines were treated with amivantamab in 96-well tissue culture plate and vehicles of test articles and culture medium were also included as negative control. The representative pictures of colonies are shown in FIGS. 6A-6B and FIGS. 7A-7B, and the IC₅₀ was determined by GraphPad.

The results showed that amivantamab can significantly inhibit the colony formation in soft agar of cell lines with high HGF expression, IM95 and JHH-5, but had limited effect on MKN45 cells with MET amplification or MHCC97H cells with pMet over-expression (FIGS. 6A-6B and FIGS. 7A-7B). These results suggest that the in vitro efficacy of amivantamab correlates with HGF expression in HCC cells, but not with the cMet over-expression or amplification.

In vivo Efficacy of Amivantamab in HCC Xenograft Models

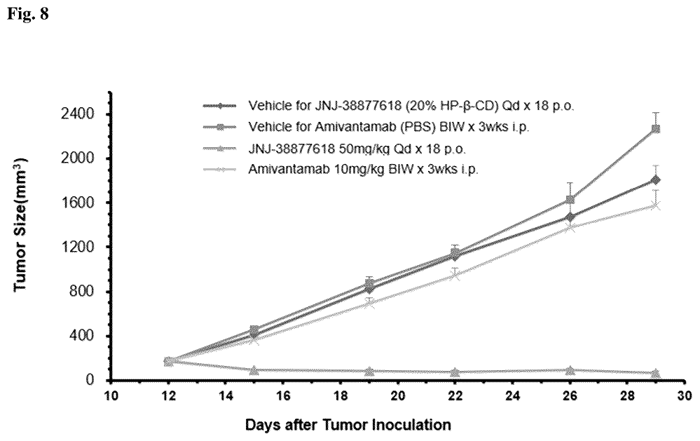

To further test the in vivo efficacy of amivantamab in tumor growth inhibition, several HCC xenograft models were tested including the xenograft models derived from HCC cell lines and patient samples. Tumor growth inhibition of MHCC97H derived xenograft model, which harbors the pMet overexpression, was tested against a small molecular cMet kinase inhibitor, JNJ-38877618, and amivantamab. As shown in FIG. 8, 10 mg/kg twice per week dosing of amivantamab had limited effect in tumor growth inhibition, while 50 mg/kg once daily dosing of JNJ-38877618 induced tumor regression in MHCC97H xenograft model.

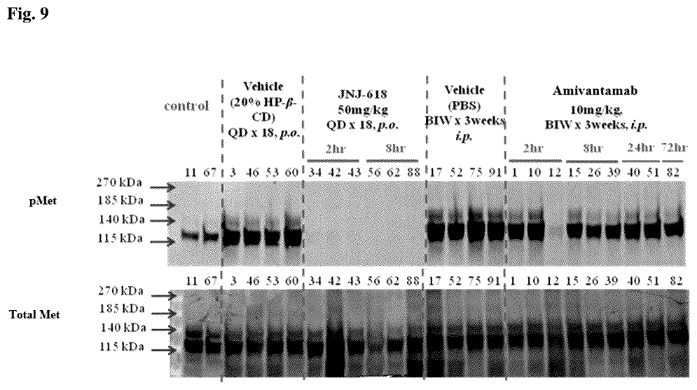

The xenograft tissues from each group were collected at the end of the study and the signal pathway modulation upon compound treatment was evaluated. As shown in FIG. 9, JNJ-38877618 significantly inhibited Met phosphorylation (pMet) but amivantamab had limited effect, which correlates with their efficacy results.

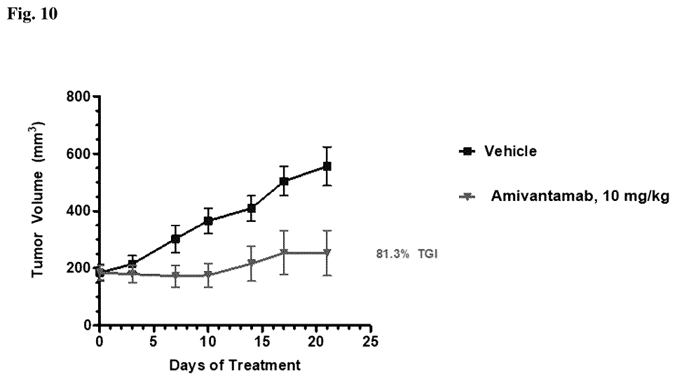

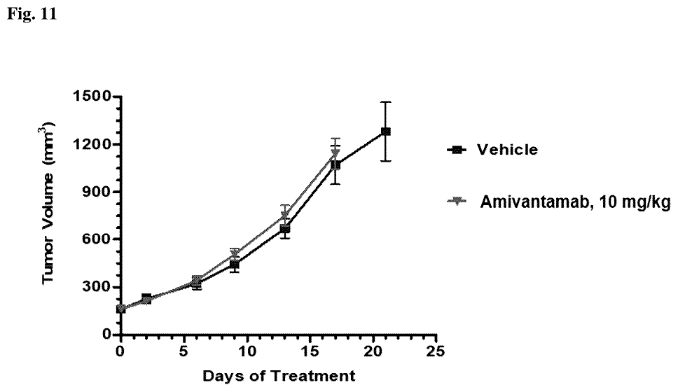

The efficacy of amivantamab in two patient derived xenograft tumor (PDX) models with high HGF expression, LI0801 and LI1646, was further explored. As shown in FIG. 10, amivantamab significantly inhibited tumor growth in LI0801 model, with 81% TGI after chronic dosing for three weeks (p=0.005). In the other PDX model, LI1646 with high HGF expression, amivantamab did not significantly inhibit tumor growth, as shown in FIG. 11. However, the LI1646 model also has a FGF19 amplification, which may act as an oncogenic driver for this model, explaining the lack of activity of amivantamab.

### Example 3. Amivantamab was Efficacious in the Inhibition of HCC PDX Tumor Growth in LI1098

To evaluate the efficacy of amivantamab, in an HCC PDX model LI1098 (CrownBio) carrying wild-type EGFR, tumor fragments from stock tumor bearing mice (passage R14P6) were harvested and inoculated into BALB/c nude mice. Each mouse was inoculated subcutaneously in the right flank with LI1098 tumor fragments (2-3 mm in diameter) for tumor development.

After establishment of palpable lesions, the tumor growth was measured twice weekly. Once the mean tumor volume reached approximate 150 mm³, animals were randomly allocated to relevant study groups with 8 mice per group. The randomization was performed according to the tumor size of each group, and the day of randomization was denoted as Day0. The treatments were started on the same day of randomization per study design in Table 6.

TABLE 6
Study Design

| | | | Dosing | Dosing | Dosing | Dosing |
|---|---|---|---|---|---|---|
| Group | N | Treatment | (mg/kg) | Route | Frequency | Schedule |
| 1 | 8 | Isotype | 10 | i.p. | BIW | Dosing 28 |
| | | Control | | | | days Keep |
| | | | | | | observing |
| | | | | | | until Day 44 |
| 2 | 8 | Amivantamab | 10 | i.p. | BIW | Dosing 28 |
| | | | | | | days Keep |
| | | | | | | observing |
| | | | | | | until Day 77 |

i.p.: intraperitoneal; BIW: twice weekly (Day 0, 3/week).

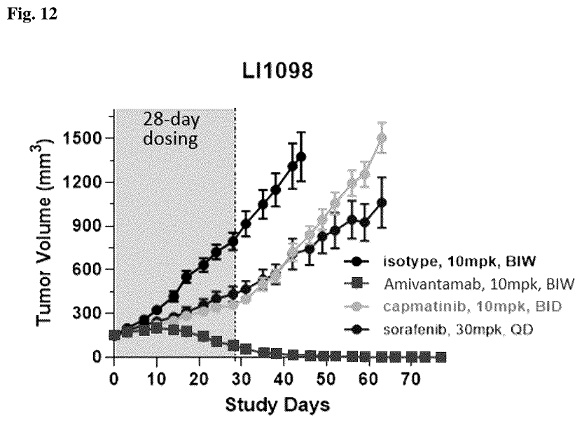

The study endpoints were to compare the tumor growth to isotype-dosing group at the end of treatments, and the subsequent tumor outgrowth after dosing stopped. The tumor size was measured twice weekly in two dimensions using a caliper, and the volumes were expressed in mm³ using the formula V=0.5×L×W², where V was tumor volume, L was tumor length (longest tumor dimension) and W was tumor width (longest tumor dimension perpendicular to L). Tumor sizes (expressed as Mean ±SEM) within the treatment groups at different time points during treatment are shown in Table 7, and the tumor growth curve over time is shown in FIG. 12.

TABLE 7
Tumor sizes during treatment with Amivantamab

| Study | Isotype control | Amivantamab |
|---|---|---|
| Days | (10 mpk, BIW) | (10 mpk, BIW) |
| 0 | 148.53 ± 5.06 | 148.51 ± 5.18 |
| 3 | 198.38 ± 10.59 | 173.59 ± 7.34 |
| 7 | 257.96 ± 17.52 | 185.84 ± 8.79 |
| 10 | 323.94 ± 21.24 | 198.40 ± 10.66 |
| 14 | 417.33 ± 35.45 | 190.41 ± 18.54 |
| 17 | 550.89 ± 41.97 | 173.11 ± 21.88 |
| 21 | 632.85 ± 46.85 | 138.96 ± 26.59 |
| 24 | 721.62 ± 50.77 | 106.32 ± 24.77 |
| 28 | 794.99 ± 59.99 | 81.00 ± 27.85 |
| 31 | 916.03 ± 87.82 | 55.76 ± 23.32 |
| 35 | 1049.58 ± 98.82 | 30.69 ± 18.17 |
| 38 | 1148.21 ± 113.57 | 25.57 ± 15.95 |
| 42 | 1311.13 ± 155.58 | 14.54 ± 14.54 |
| 44 | 1375.07 ± 168.80 | |
| 45 | | 13.77 ± 13.77 |
| 49 | | 6.23 ± 6.23 |
| 52 | | 5.92 ± 5.92 |
| 56 | | 5.20 ± 5.20 |
| 59 | | 0.00 ± 0.00 |
| 63 | | 0.00 ± 0.00 |
| 66 | | 0.00 ± 0.00 |
| 70 | | 0.00 ± 0.00 |
| 73 | | 0.00 ± 0.00 |
| 77 | | 0.00 ± 0.00 |

a: Data represent as Mean tumor volume ± SEM (standard error).

Tumor growth inhibition (TGI%) was an indication of anti-tumor activities, and calculated as TGI%=((C−C0)−(T−T0))/(C−C0)×100%, T and C were the mean tumor volume (TV) of treated and control groups, respectively, on the day when dosing stopped (on Day28). Tumor growth inhibition is summarized in Table 8. To compare the mean tumor volumes of treatment groups with the control group, we first used Bartlett's test to check the assumption of homogeneity of variance across all groups (p<0.05), and then ran Kruskal-Wallis test for overall equality of medians among all groups (p<0.05). Post hoc testing was further performed by running Conover's non-parametric test with single-step p-value adjustment for all pairwise comparisons. Here, ap-value <0.05 was considered to be statistically significant. The body weight changes were monitored twice weekly.

TABLE 8
Tumor volume and growth inhibition

| | Tumor Volume^{a} | Tumor Volume | | |
|---|---|---|---|---|
| | (mm³) on | (mm³) on | | |
| Treatment | Day 0 | Day 21 | TGI %^{b} | P value^{c} |
| Isotype | 148.53 ± | 794.99 ± | — | — |
| Control | 5.06 | 59.99 | | |
| Amivantamab | 148.51 ± | 81.00 ± | 110.44% | 4.84 × 10⁻¹³ |
| | 5.18 | 27.85 | | (***) |

^{a}Data represent Mean tumor volume ± SEM.
^{b}TGI % calculated as below:
$TGI \% = \frac{( C 28 - C 0 ) - ( T 28 - T 0 )}{( C 28 - C 0 )} \times 100 \%$ C (28 or 0): Mean tumor volume of Control group on indicated Study Day.
T (28 or 0): Mean tumor volume of Treatment group on indicated Study Day.
^{c}P-value were calculated by performing Conover's non-parametric many-to-one comparison test. 0 here represents the calculation threshold for getting an exact p-value.

As shown in FIG. 12, amivantamab (10 mg/kg BIW) showed pronounced anti-tumor activity (110.44% on Day 28) during treatments. Tumors were continuously regressed after dosing stopped. Complete tumor regressions appeared on Day 59, and no tumor relapsed until study end (Day 77).

### Example 4. Amivantamab was Efficacious in the Inhibition of HCC PDX Tumor Growth in LI1037

To evaluate the efficacies of amivantamab, in an HCC PDX model LI1037 (CrownBio) carrying wild-type EGFR, tumor fragments from stock tumor bearing mice (passage R14P8) were harvested and inoculated into BALB/c nude mice. Each mouse was inoculated subcutaneously in the right flank with LI1037 tumor fragments (2-3 mm in diameter) for tumor development.

After establishment of palpable lesions, the tumor growth was measured twice weekly. Once the mean tumor volume reached approximate 150 mm³, animals were randomly allocated to relevant study groups with 8 mice per group. The randomization was performed according to the tumor size of each group, and the day of randomization was denoted as Day0. The treatments were started on the same day of randomization per study design in Table 9.

TABLE 9
Study Design

| | | | Dosing | Dosing | Dosing | Dosing |
|---|---|---|---|---|---|---|
| Group | N | Treatment | (mg/kg) | Route | Frequency | Schedule |
| 1 | 8 | Isotype | 10 | i.p. | BIW | Dosing 21 |
| | | Control | | | | days |
| 2 | 8 | Amivantamab | 10 | i.p. | BIW | Dosing 28 |
| | | | | | | days Keep |
| | | | | | | observing |
| | | | | | | until Day 77 |

i.p.: intraperitoneal; BIW: twice weekly (Day 0, 3/week).

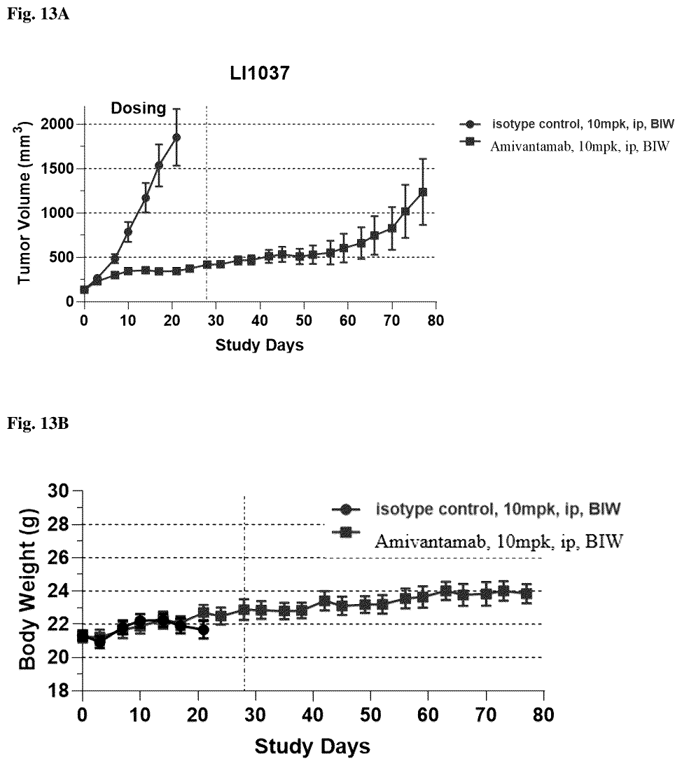

The study endpoints were to compare the tumor growth to isotype-dosing group at the end of treatments, and the subsequent tumor outgrowth after dosing stopped. The tumor size was measured twice weekly in two dimensions using a caliper, and the volumes were expressed in mm³ using the formula V=0.5×L×W², where V was tumor volume, L was tumor length (longest tumor dimension), and W was tumor width (longest tumor dimension perpendicular to L). Tumor sizes (expressed as Mean±SEM) within the treatment groups at different time points during treatment are shown in Table 10, and the tumor growth curve over time is shown in FIG. 13A.

TABLE 10
Tumor sizes during treatment with amivantamab

| Study | Isotype control | Amivantamab |
|---|---|---|
| Days | (10 mpk, BIW) | (10 mpk, BIW) |
| 0 | 134.5 ± 8.12 | 134.52 ± 8.58 |
| 3 | 265.88 ± 15.61 | 227.65 ± 16.79 |
| 7 | 483.38 ± 49.64 | 299.12 ± 17.47 |
| 10 | 785.57 ± 111.36 | 343.82 ± 36.47 |
| 14 | 1171.36 ± 169.11 | 353.11 ± 26.93 |
| 17 | 1535.74 ± 237.42 | 340.42 ± 28.69 |
| 21 | 1850.71 ± 319.03 | 344.18 ± 30.99 |
| 24 | | 371.99 ± 28.62 |
| 28 | | 416.98 ± 37.28 |
| 31 | | 420.28 ± 39.43 |
| 35 | | 462.43 ± 46.65 |
| 38 | | 469.00 ± 53.03 |
| 42 | | 509.59 ± 74.91 |
| 45 | | 531.91 ± 85.73 |
| 49 | | 507.69 ± 88.35 |
| 52 | | 527.25 ± 105.71 |
| 56 | | 552.48 ± 134.94 |
| 59 | | 602.41 ± 161.95 |
| 63 | | 658.90 ± 179.60 |
| 66 | | 744.32 ± 217.44 |
| 70 | | 827.07 ± 243.32 |
| 73 | | 1017.55 ± 300.94 |
| 77 | | 1235.40 ± 372.85 |

a: Data represent as Mean tumor volume ± SEM (standard error).

Tumor growth inhibition (TGI%) was an indication of anti-tumor activities, and calculated as TGI%=((C−C0)−(T−T0))/(C−C0)×100%, T and C were the mean tumor volume (TV) of treated and control groups, respectively on the day when mean TV of control group reached humane endpoints (approximate to 2000 mm³, on Day21). Tumor growth inhibition is summarized in Table 11. To compare the mean tumor volumes of treatment groups with control group, we first used Bartlett's test to check the assumption of homogeneity of variance across all groups (p<0.05), and then ran Kruskal-Wallis test for overall equality of medians among all groups (p<0.05). Post hoc testing was further performed by running Conover's non-parametric test with single-step p-value adjustment for all pairwise comparisons. Here, a p-value <0.05 was considered to be statistically significant. The body weight changes were monitored twice weekly and also shown in FIG. 13B.

TABLE 11
Tumor volume and growth inhibition

| | Tumor Volume^{a} | Tumor Volume | | |
|---|---|---|---|---|
| | (mm³) on | (mm³) on | | |
| Treatment | Day 0 | Day 21 | TGI %^{b} | P value^{c} |
| Isotype | 134.5 ± | 1850.71 ± | — | — |
| Control | 8.12 | 319.03 | | |
| Amivantamab | 134.52 ± | 344.18 ± | 87.78% | 4.56 × 10⁻⁸ |
| | 8.58 | 30.99 | | (***) |

^{a}Data represent Mean tumor volume ± SEM.
^{b}TGI % calculated as below:
$TGI \% = \frac{( C 21 - C 0 ) - ( T 21 - T 0 )}{( C 21 - C 0 )} \times 100 \%$ C (21 or 0): Mean tumor volume of Control group on indicated Study Day.
T (21 or 0): Mean tumor volume of Treatment group on indicated Study Day.
^{c}P-value were calculated by performing Conover's non-parametric many-to-one comparison test. 0 here represents the calculation threshold for getting an exact p-value.

As shown in FIG. 13A, amivantamab (10 mg/kg BIW) showed robust anti-tumor activity (87.78% on Day21) during treatments. Tumors were relapsed slowly after dosing stopped, and tumor volume reached 1235.40±372.85 mm³ on Day77.

### Example 5. Amivantamab was Efficacious in the Inhibition of HCC PDX Tumor Growth in LI0801

To evaluate the efficacies of amivantamab, in an HCC PDX model LI0801 (CrownBio) carrying wild-type EGFR, tumor fragments from stock tumor bearing mice (passage R20P6) were harvested and inoculated into BALB/c nude mice. Each mouse was inoculated subcutaneously in the right flank with LI0801 tumor fragments (2-3 mm in diameter) for tumor development.

After establishment of palpable lesions, the tumor growth was measured twice weekly. Once the mean tumor volume reached approximate 150 mm³, animals were randomly allocated to relevant study groups with 8 mice per group. The randomization was performed according to the tumor size of each group, and the day of randomization was denoted as Day0. The treatments were started on the same day of randomization per study design in Table 12.

TABLE 12
Study Design

| | | | Dosing | Dosing | Dosing | Dosing |
|---|---|---|---|---|---|---|
| Group | N | Treatment | (mg/kg) | Route | Frequency | Schedule |
| 1 | 8 | Isotype | 10 | i.p. | BIW | Dosing 21 |
| | | Control | | | | days |
| 2 | 8 | Amivantamab | 10 | i.p. | BIW | Dosing 28 |
| | | | | | | days Keep |
| | | | | | | observation |
| | | | | | | till Day 42 |

i.p.: intraperitoneal; BIW: twice weekly (Day 0, 3/week).

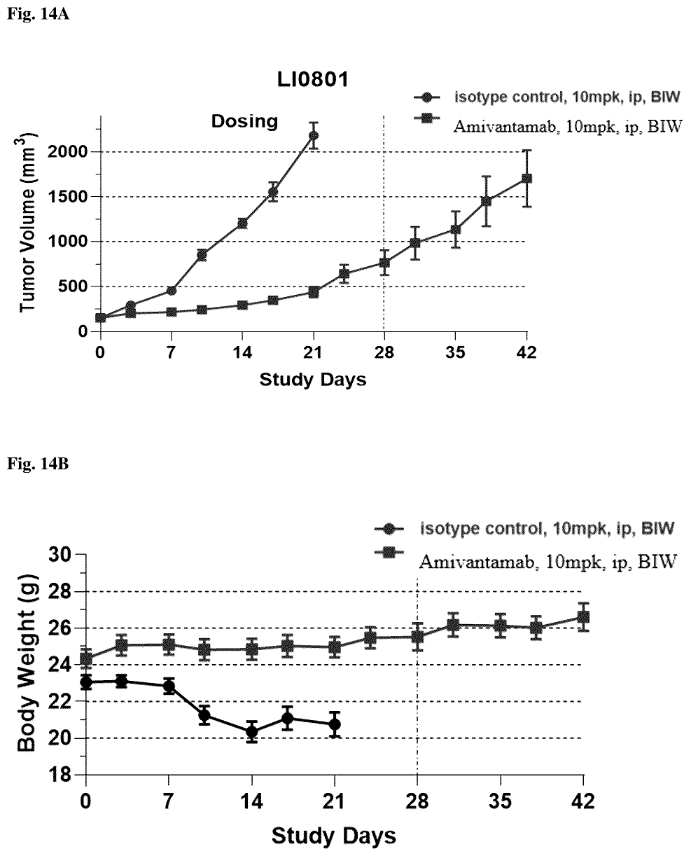

The study endpoints were to compare the tumor growth to isotype-dosing group at the end of treatments, and the subsequent tumor outgrowth after dosing stopped. The tumor size was measured twice weekly in two dimensions using a caliper, and the volumes were expressed in mm³ using the formula V=0.5×L×W², where V was tumor volume, L was tumor length (longest tumor dimension), and W was tumor width (longest tumor dimension perpendicular to L). Tumor sizes (expressed as Mean ±SEM) within the treatment groups at different time points during treatment are shown in Table 13, and the tumor growth curve over time is shown in FIG. 14A.

TABLE 13
Tumor sizes during treatment with Amivantamab

| Study | Isotype control | Amivantamab |
|---|---|---|
| Days | (10 mpk, BIW) | (10 mpk, BIW) |
| 0 | 150.54 ± 6.70 | 150.57 ± 7.18 |
| 3 | 294.27 ± 20.07 | 202.38 ± 14.65 |
| 7 | 453.02 ± 39.68 | 215.59 ± 15.82 |
| 10 | 850.29 ± 59.66 | 243.6 ± 19.76 |
| 14 | 1204.73 ± 53.22 | 293.09 ± 29.10 |
| 17 | 1555.38 ± 105.99 | 346.53 ± 41.59 |
| 21 | 2180.93 ± 144.29 | 438.12 ± 60.03 |
| 24 | | 641.22 ± 102.43 |
| 28 | | 766.04 ± 138.89 |
| 31 | | 982.34 ± 184.02 |
| 35 | | 1135.8 ± 204.82 |
| 38 | | 1449.02 ± 276.93 |
| 42 | | 1703.12 ± 316.09 |

a: Data represent as Mean tumor volume ± SEM (standard error).

Tumor growth inhibition (TGI%) was an indication of anti-tumor activities, and calculated as TGI%=((C−C0)−(T−T0))/(C−C0)×100%, T and C were the mean tumor volume (TV) of treated and control groups, respectively on the day when mean TV of control group reached humane endpoints (approximate to 2000 mm³, on Day21). Tumor growth inhibition is summarized in Table 14. To compare the mean tumor volumes of treatment groups with control group, we first used Bartlett's test to check the assumption of homogeneity of variance across all groups (p<0.05), and then ran Kruskal-Wallis test for overall equality of medians among all groups (p<0.05). Post hoc testing was further performed by running Conover's non-parametric test with single-step p-value adjustment for all pairwise comparisons. Here, a p-value <0.05 was considered to be statistically significant. The body weight changes were monitored twice weekly and also shown in FIG. 14B.

TABLE 14
Tumor volume and growth inhibition

| | Tumor Volume^{a} | Tumor Volume | | |
|---|---|---|---|---|
| | (mm³) on | (mm³) on | | |
| Treatment | Day 0 | Day 21 | TGI %^{b} | P value^{c} |
| Isotype | 150.54 ± | 2180.93 ± | — | — |
| Control | 6.70 | 144.29 | | |
| Amivantamab | 150.57 ± | 438.12 ± | 85.86% | 8.39 × 10⁻⁴ |
| | 7.18 | 60.03 | | (***) |

^{a}Data represent Mean tumor volume ± SEM.
^{b}TGI % calculated as below:
$TGI \% = \frac{( C 21 - C 0 ) - ( T 21 - T 0 )}{( C 21 - C 0 )} \times 100 \%$ C (21 or 0): Mean tumor volume of Control group on indicated Study Day.
T (21 or 0): Mean tumor volume of Treatment group on indicated Study Day.
^{c}P-value were calculated by performing Conover's non-parametric many-to-one comparison test. 0 here represents the calculation threshold for getting an exact p-value.

As shown in FIG. 14A, amivantamab (10 mg/kg BIW) showed robust anti-tumor activity (85.86% on Day21) during treatments. Tumors were relapsed rapidly after dosing stopped, and tumor volumes reached 1703.12±316.09 mm³ on Day42.

The experiments of the present disclosure demonstrated that amivantamab inhibited in vitro and in vivo cell growth in HCC cell lines with HGF over-expression, supporting amivantamab clinic trial assessments in HCC patients with high HGF expression.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of treating liver cancer in a subject in need thereof, comprising administering to the subject a therapeutically effective amount of a bispecific anti-epidermal growth factor receptor (EGFR)/hepatocyte growth factor receptor (c-Met) antibody.

2. The method of claim 1, wherein the liver cancer is hepatic cellular carcinoma (HCC).

3. The method of claim 1, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

4. The method of claim 3, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

5. The method of claim 3, wherein the bispecific anti-EGFR/c-Met antibody is an IgG1 isotype.

6. The method of claim 3, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

7. The method of claim 1, wherein the bispecific anti-EGFR/c-Met antibody comprises a biantennary glycan structure with a fucose content of about between 1% to about 15%.

8. The method of claim 1, wherein the bispecific anti-EGFR/c-Met antibody is administered intravenously to the subject.

9. The method of claim 8, wherein the bispecific anti-EGFR/c-Met antibody is administered at a dose of between about 350 mg to about 1400 mg.

10. The method of claim 9, wherein the bispecific anti-EGFR/c-Met antibody is administered at a dose of about 350 mg, 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg or 1400 mg.

11. The method of claim 10, wherein the bispecific anti-EGFR/c-Met antibody is administered at a dose of 1050 mg.

12. The method of claim 10, wherein the bispecific anti-EGFR/c-Met antibody is administered at a dose of 1400 mg.

13. The method of claim 1, wherein the bispecific anti-EGFR/c-Met antibody is administered subcutaneously or intradermally to the subject.

14. The method of claim 13, wherein the bispecific anti-EGFR/c-Met antibody is administered subcutaneously or intradermally at a dose sufficient to achieve a therapeutic effect in the subject.

15. The method of claim 1, wherein the bispecific anti-EGFR/c-Met antibody is administered twice a week, once a week, once in two weeks, once in three weeks or once in four weeks.

16. The method of claim 1, wherein the bispecific anti-EGFR/c-Met antibody is administered twice a week, once a week, once in two weeks, once in three weeks or once in four weeks.

17. The method of claim 16, wherein the bispecific anti-EGFR/c-Met antibody is administered once a week for four weeks and once in two weeks thereafter.

18. The method of claim 1, wherein the subject has received a prior treatment.

19. The method of claim 18, wherein the prior treatment comprises a multi-targeted kinase inhibitor (MKI), an immunotherapy, anti-VEGF/VEGFR therapy, or a combination thereof.

20. The method of claim 18, wherein the prior treatment comprises a multi-targeted kinase inhibitor (MKI).

21. The method of claim 20, wherein the multi-targeted kinase inhibitor (MKI) is sorafenib regorafenib, lenvatinib, cabozantinib, apatinib, or a combination thereof.

22. The method of claim 19, wherein the immunotherapy comprises a PD-(L)1 axis inhibitor, or a CTLA-4 inhibitor.

23. The method of claim 22, wherein the PD-(L)1 axis inhibitor comprises atezolizumab, nivolumab, pembrolizumab, camrelizumab, tislelizumab, or a combination thereof.

24. The method of claim 22, wherein the CTLA-4 inhibitor comprises ipilimumab.

25. The method of claim 19, wherein the anti-VEGF/VEGFR therapy comprises bevacizumab or ramucirumab.

26. The method of claim 1, wherein the subject is treatment naive.


SEQUENCE LISTING (20 sequences)

SEQ ID NO: 1 - protein, 5 aa, Homo sapiens
TYGMH

SEQ ID NO: 2 - protein, 17 aa, Homo sapiens
VIWDDGSYKYYGDSVKG

SEQ ID NO: 3 - protein, 16 aa, Homo sapiens
DGITMVRGVMKDYFDY

SEQ ID NO: 4 - protein, 11 aa, Homo sapiens
RASQDISSALV

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 5 aa, Homo sapiens
SYGIS

SEQ ID NO: 8 - protein, 17 aa, Homo sapiens
WISAYNGYTNYAQKLQG

SEQ ID NO: 9 - protein, 10 aa, Homo sapiens
DLRGTNYFDY

SEQ ID NO: 10 - protein, 11 aa, Homo sapiens
RASQGISNWLA

SEQ ID NO: 11 - protein, 7 aa, Homo sapiens
AASSLLS

SEQ ID NO: 12 - protein, 9 aa, Homo sapiens
QQANSFPIT

SEQ ID NO: 13 - protein, 125 aa, Homo sapiens [sequence omitted]

SEQ ID NO: 14 - protein, 107 aa, Homo sapiens [sequence omitted]

SEQ ID NO: 15 - protein, 119 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Homo sapiens
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Homo sapiens
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC